(19) United States
(12) Patent Application Publication
(10) Pub. No.: US 2023/0348905 A1
(43) Pub. Date: Nov. 2, 2023

(54) METHODS FOR THE REDUCTION OF Z-AAT PROTEIN LEVELS

(71) Applicant: Arrowhead Pharmaceuticals, Inc., Pasadena, CA (US)

(72) Inventors: Dawn Christianson, Solana Beach, CA (US); James C. Hamilton, Arcadia, CA (US); Bruce Given, Charleston, SC (US)

(73) Assignee: Arrowhead Pharmaceuticals, Inc., Pasadena, CA (US)

(21) Appl. No.: 18/026,340

(22) PCT Filed: Sep. 14, 2021
(86) PCT No.: PCT/US2021/050247

§ 371 Date: Mar. 14, 2023

Related U.S. Application Data
(60) Provisional application No. 63/180,487, filed on Apr. 27, 2021.
(60) Provisional application No. 63/078,658, filed on Sep. 15, 2020.

(51) Int. Cl. A61P 1/16; C12N 15/113

(52) U.S. Cl. CPC C12N 15/113; A61P 1/16; C12N 2310/14; C12N 2320/35

(57) ABSTRACT

Described are methods of reducing liver Z-AAT protein levels in a human subject with a PiZZ genotype of alpha-1 antitrypsin (AAT) by using pharmaceutical compositions that include AAT RNAi agents. The pharmaceutical compositions disclosed herein that include AAT RNAi agents, when administered to a human subject with a PiZZ mutation, lead to a reduction in liver Z-AAT protein levels, including both soluble and insoluble Z-AAT protein. Such reductions can lead to the treatment of liver diseases associated with AAT deficiency such as chronic hepatitis, cirrhosis, increased risk of hepatocellular carcinoma, transaminitis, cholestasis, fibrosis, fulminant hepatic failure, and other liver-related diseases.

### CROSS-REFERENCE TO RELATED APPLICATIONS

This PCT application claims the priority benefit of U.S. Provisional Application Nos. 63/078,658, filed on Sep. 15, 2020, and 63/180,487, filed Apr. 27, 2021, both of which are herein incorporated by reference in their entireties.

### REFERENCE TO SEQUENCE LISTING SUBMITTED ELECTRONICALLY

The content of the electronically submitted sequence listing in ASCII text file (Name 3817_084PC02_SequenceListing.txt; Size: 5,721 bytes; and Date of Creation: Sep. 12, 2021) filed with the application is incorporated herein by reference in its entirety.

### FIELD

Disclosed herein are methods for the reduction of liver Z-AAT protein levels in human subjects with the Z mutation of alpha-1 antitrypsin deficiency (AATD) using pharmaceutical compositions that include RNA interference (RNAi) agents that inhibit alpha-1 antitrypsin gene expression.

### BACKGROUND

Alpha-1 antitrypsin (AAT, α1-antitrypsin, or A1AT) is a protease inhibitor belonging to the serpin superfamily encoded in humans by the SERPINA1 gene. Normal AAT protein is a circulating glycoprotein protease inhibitor primarily synthesized in the liver by hepatocytes and secreted into the blood. The known physiologic function of AAT is to inhibit neutrophil proteases, which serves to protect host tissues from non-specific injury during periods of inflammation.

AATD is an autosomal, codominant genetic disorder that results in low circulating levels of AAT and causes early pulmonary disease in adults and liver disease in children and adults. The prevalence range of AATD is about 1 in every 1,500 to 5,000 individuals and most often affects persons with European ancestry.

The most clinically significant and serious form of AATD is caused by homozygosity for the Z mutation (referred to as the PiZZ genotype), where a single nucleotide polymorphism encoding a glutamic acid is substituted with a lysin at position 342 of the mature protein (Glu342Lys). The Z mutant allele, through a single point mutation, renders the mutant Z form AAT protein (the “Z-AAT protein”) prone to abnormal folding causing intracellular retention in the endoplasmic reticulum (ER) of hepatocytes. Other rarer mutations also result in misfolded accumulated protein in hepatocytes. The mutant Z-AAT protein monomers are able to amass into polymer aggregates, which are sometimes referred to as “globules.” The polymeric Z-AAT stress the ER and trigger a cycle of continuous hepatocyte injury and healing, leading to fibrosis, cirrhosis, and increased risk of hepatocellular carcinoma. Further, the absence of circulating anti-protease activity leaves the lung vulnerable to injury by neutrophil elastase, particularly in the setting of lung inflammation, resulting in the development of respiratory complications such as emphysema or other pulmonary disease.

Individuals with the homozygous PiZZ genotype have severe deficiency of functional AAT. Weekly use of AAT augmentation therapy, using purified human AAT, helps prevent lung damage in affected individuals. Such currently marketed products include, for example, PROLASTIN®-C, PROLASTIN®, GLASSIA™, ARALAST® NP, and ZEMAIRA®. However, while the administration of purified AAT can ameliorate or help prevent lung damage caused by the absence or low levels of endogenously secreted AAT, AATD patients (with an AAT-mutation that results in polymer formation) remain vulnerable to endoplasmic reticulum liver storage disease caused by the deposition and accumulation of excessive abnormally folded AAT protein. Accumulated Z-AAT protein in a “globule” conformation in hepatocytes is a well-known histologic characteristic of AATD liver disease and is believed to lead to proteotoxic effects that are responsible for inducing liver injury, including liver cell damage and death and chronic liver injury in individuals with AATD (see, e.g., D. Lindblad et al., Hepatology 2007, 46: 1228-1235). It has been reported that null/null patients, who produce no AAT, develop severe pulmonary disease but have normal liver morphology, providing evidence that the accumulation of the mutant AAT, and not the lack of circulating AAT, leads to hepatic disease (Feldman, G. et al, The Ultrastructure of Hepatocytes in alpha-1 antitrypsin deficiency with genotype Pi_, Gut. 1975; 16:796-799).

AATD predisposes individuals to liver disease in children and adults and to early-onset emphysema in adults. Patients with AATD often develop liver disease, which can be severe or fatal, even in infancy. While some patients with AATD escape detection initially, eventually fibrosis accumulates and leads to clinically apparent liver disease. Clinical presentations of injury in the liver include chronic hepatitis, cirrhosis, increased risk of hepatocellular carcinoma, transaminitis, cholestasis, fibrosis, and even fulminant hepatic failure.

Z-AAT protein accumulation in hepatocytes has been clearly identified as the cause of progressive liver disease in AATD patients. Elimination of mutant protein accumulation in hepatocytes may halt the progression of liver disease. Removal of the mutant protein insult may also allow for regression of already present fibrosis. There is currently no clinically approved treatment to prevent the onset, slow the progression, or otherwise treat liver disease caused by AATD.

RNAi agents have emerged as a promising avenue for treating AATD patients. Dosing strategies are an important consideration in the treatment of AATD with RNAi agents. Less frequent dosing is valued by patients, leads to increased compliance, and smaller dosing amounts can be advantageous in the overall safety profile of the drug. There thus exists a need for a low dose, infrequent method for the treatment of AATD.

### SUMMARY

Described herein are methods of reducing liver Z-AAT protein levels in human subjects with the Z mutation of AATD. In one aspect, the methods comprise administering to the human subject a pharmaceutical composition that includes the composition described in Table 2 (i.e., AAT RNAi Drug Substance, also referred to herein as ADS-001, or a salt thereof), at a dose of between about 5 mg and about 300 mg of the AAT RNAi Drug Substance (e.g., ADS-001, or a salt thereof, wherein the pharmaceutical composition is administered, e.g., subcutaneously and there is, e.g., about one month or about four weeks between doses. In some embodiments, the pharmaceutical composition used in the methods disclosed herein comprises, consists of, or consists essentially of the Formulated AAT RNAi Drug Substance as described in Table 3.1 (also referred to herein as ADS-001-1, or a salt thereof) or the formulation of Table 3.2 (also referred to herein as ADS-001-2). As used herein and depending on the context, the term “about” or “approximately” means within 5%, e.g., within 5%, 4%, 3%, 2%, or 1% of a given value or range.

Additionally, described herein are methods of reducing Z-AAT liver protein levels in a human subject with the Z mutation of AATD, the methods comprising administering to the human subject a pharmaceutical composition that includes the AAT RNAi Drug Substance as described in Table 2 (e.g., ADS-001, or a salt thereof) at a dose of between about 5 mg and about 200 mg, wherein the pharmaceutical composition is administered, e.g., subcutaneously and there, e.g., is at least about one month between dose administrations (i.e., about monthly dosing).

Further described herein are methods of treating AATD in a human subject in need thereof, the methods comprising administering to the human subject a pharmaceutical composition that includes the AAT RNAi Drug Substance as described in Table 2 (e.g., ADS-001, or a salt thereof) at a dose of between about 5 mg and about 300 mg, wherein the pharmaceutical composition is administered, e.g., subcutaneously and there is, e.g., about three months between dose administrations (i.e., quarterly dosing).

Also described herein are methods of treating AATD in a human subject in need thereof, the methods comprising administering to the human subject a pharmaceutical composition that includes the AAT RNAi Drug Substance as described in Table 2 (e.g., ADS-001 or a salt thereof) at a dose of between about 5 mg and about 200 mg, wherein the pharmaceutical composition is administered, e.g., subcutaneously and there is, e.g., about three months between dose administrations (i.e., quarterly dosing).

Described herein are methods of treating AATD in a human subject in need thereof, the methods comprising administering to the human subject a pharmaceutical composition that includes the AAT RNAi Drug Substance as described in Table 2 (e.g., ADS-001 or a salt thereof) at a dose of between about 5 mg and about 300 mg, wherein the pharmaceutical composition is administered, e.g., subcutaneously, and wherein the initial dose is followed, e.g., by a second dose about four weeks or about one month later, and thereafter for subsequent doses there is, e.g., about three months between dose administrations.

Described herein are methods of treating AATD in a human subject in need thereof, the methods comprising administering to the human subject a pharmaceutical composition that includes the AAT RNAi Drug Substance as described in Table 2 (e.g., ADS-001 or a salt thereof) at a dose of between about 5 mg and about 200 mg, wherein the pharmaceutical composition is administered, e.g., subcutaneously, and wherein the initial dose is followed, e.g., by a second dose about one month later, and thereafter for subsequent doses there is, e.g., about three months between dose administrations.

In some embodiments, the dose of AAT RNAi Drug Substance (e.g., ADS-001 or a salt thereof) administered in each dose is, e.g., between about 25 mg and about 200 mg. In some embodiments, the dose of AAT RNAi Drug Substance (e.g., ADS-001 or a salt thereof) administered in each dose is between about 100 mg and about 200 mg. In some embodiments, the dose of AAT RNAi Drug Substance (e.g., ADS-001 or a salt thereof) administered in each dose is about 100 mg. In some embodiments, the dose of AAT RNAi Drug Substance (e.g., ADS-001 or a salt thereof) administered in each dose is about 200 mg. In some embodiments, the dose of AAT RNAi Drug Substance administered in each dose is no greater than 200 mg.

The treatment methods disclosed herein can slow or halt the progression of liver disease in a human subject having AATD, which can allow for fibrotic tissue repair. The methods disclosed herein can, in some embodiments, treat AATD liver diseases including fibrosis, cirrhosis, increased risk of hepatocellular carcinoma, chronic hepatitis, transaminitis, cholestasis, fulminant hepatic failure, and other liver-related conditions and diseases caused by AATD. In some aspects, the methods disclosed herein can prevent, delay the onset, or ameliorate the symptoms, complications, and/or sequelae of AATD liver diseases disclosed herein.

The pharmaceutical compositions that include AAT RNAi agents disclosed herein (e.g., ADS-001 or a salt thereof) can be administered to a human subject to inhibit the expression of the alpha-1 antitrypsin gene in the subject. In some embodiments, the subject is a human that has been previously diagnosed with having AATD.

Another aspect of the invention provides for the use of the AAT RNAi Drug Substance described in Table 2 (e.g., ADS-001 or a salt thereof) for the treatment of alpha-1 antitrypsin deficiency (AATD) in a human subject in need thereof, wherein the use comprises administering to the subject a pharmaceutical composition that comprises the AAT RNAi Drug Substance described in Table 2 (e.g., ADS-001 or a salt thereof) at a dose of between about 5 mg to about 300 mg of the AAT RNAi Drug Substance, wherein the pharmaceutical composition is administered once each month, e.g., by subcutaneous injection.

Another aspect of the invention provides for the use of the AAT RNAi Drug Substance described in Table 2 (e.g., ADS-001 or a salt thereof) for the treatment of alpha-1 antitrypsin deficiency (AATD) in a human subject in need thereof, wherein the use comprises administering to the subject a pharmaceutical composition that comprises the AAT RNAi Drug Substance described in Table 2 (e.g., ADS-001 or a salt thereof) at a dose of between about 5 mg to about 300 mg of the AAT RNAi Drug Substance, wherein the pharmaceutical composition is administered once every three months, e.g., by subcutaneous injection.

In some aspects, the present disclosure provides a method of reducing liver Z-AAT protein levels in a human subject with a PiZZ genotype of alpha-1 antitrypsin, the method comprising (i) administering to the subject an initial dose of a pharmaceutical composition that comprises the AAT RNAi Drug Substance described in Table 2 at a dose of between about 5 mg to about 300 mg of the AAT RNAi Drug Substance, (ii) administering to the subject a second dose of the pharmaceutical composition about four weeks or about one month after the initial dose, and (iii) administering to the subject a third dose of the pharmaceutical composition about twelve weeks or about three months after the second dose, wherein the doses are administered by subcutaneous injection.

In some aspects, the dose of the AAT RNAi Drug Substance is between about 25 mg and about 300 mg. In some aspects, the dose of the AAT RNAi Drug Substance is between about 25 mg and about 200 mg. In some aspects, the dose of the AAT RNAi Drug Substance is between about 100 mg and about 200 mg. In some aspects, the dose of the AAT RNAi Drug Substance is about 100 mg. In some aspects, the dose of the AAT RNAi Drug Substance is about 200 mg. In some aspects, the dose of the AAT RNAi Drug Substance is about 200 mg or less. In some aspects, soluble liver Z-AAT protein level is reduced. In some aspects, insoluble liver Z-AAT protein level is reduced. In some aspects, both insoluble liver Z-AAT protein level and soluble liver Z-AAT protein level is reduced.

In some aspects, the method further comprises administering additional doses after the third dose, wherein the additional doses are administered about every twelve weeks or about every three months thereafter. In some aspects, the liver Z-AAT protein level is reduced within six months from the initial dose. In some aspects, the liver Z-AAT protein level is reduced within about one year from the initial dose. In some aspects, the Z-AAT protein level is reduced after the administration of only three doses of AAT RNAi Drug Substance. In some aspects, the liver shows no worsening or an improvement in fibrosis. In some aspects, liver enzymes of ALT (alanine aminotransferase), GGT (γ-glutamyl transferase), or both are reduced. In some aspects, fibrogenesis marker Pro-C3 is reduced. In some aspects, portal liver inflammation is reduced. In some aspects, non-invasive measurement of liver stiffness by transient elastography (FIBROSCAN®) is improved.

In some aspects, the subject is further administered an additional therapeutic for the treatment of AATD. In some aspects, the subject is further administered a therapeutic for the treatment of lung damage, emphysema, or other lung diseases or disorders caused by the deficiency of endogenously secreted AAT protein. In some aspects, the additional therapeutic comprises human AAT protein, purified human alpha-1 proteinase inhibitor, or recombinant AAT protein.

In some aspects, the pharmaceutical composition that comprises the AAT RNAi Drug Substance described in Table 2 is packaged in a kit, container, pack, dispenser, pre-filled syringe, or vials. In some aspects, the pharmaceutical composition comprises, consists of, or consists essentially of the Formulated AAT RNAI Drug Substance described in Table 3.1 or Table 3.2. In some aspects, the administration of one or more doses of the pharmaceutical composition is performed by the subject. In some aspects, the administration of one or more doses of the pharmaceutical composition is performed by a medical professional. In some aspects, the subject is an adult.

The present disclosure also provides a method of treating AATD in a human subject with a PiZZ genotype of alpha-1 antitrypsin, the method comprising (i) administering to the subject an initial dose of a pharmaceutical composition that comprises the AAT RNAi Drug Substance described in Table 2 at a dose of between about 5 mg to about 300 mg of the AAT RNAi Drug Substance, (ii) administering to the subject a second dose of the pharmaceutical composition about four weeks or about one month after the initial dose, and (iii) administering to the subject a third dose of the pharmaceutical composition about twelve weeks or about three months after the second dose, wherein the doses are administered by subcutaneous injection. In some aspects, the condition or disease caused by AATD is a liver disease. In some aspects, the liver disease is chronic hepatitis, cirrhosis, increased risk of hepatocellular carcinoma, transaminitis, cholestasis, fibrosis, or fulminant hepatic failure. In some aspects, the dose of the AAT RNAi Drug Substance is between about 100 mg and about 200 mg. In some aspects, the dose of the AAT RNAi Drug Substance is about 200 mg or less.

In some aspects of the methods disclosed herein, monomer (soluble) liver Z-AAT protein level is reduced. In some aspects, insoluble liver Z-AAT protein level is reduced. In some aspects, both insoluble liver Z-AAT protein level and soluble liver Z-AAT protein level is reduced. In some aspects of the method of treating AATD in a human subject with a PiZZ genotype of alpha-1 antitrypsin disclosed herein, the method further comprises administering additional doses after the third dose, wherein the additional doses are administered about every twelve weeks or about every three months thereafter. In some aspects, the liver Z-AAT protein level is reduced within about six months of the initial dose. In some aspects, the liver Z-AAT protein level is reduced within about one year of the initial dose. In some aspects, the Z-AAT protein level is reduced after the administration of only three doses of AAT RNAi Drug Substance.

In some aspects of the methods disclosed herein, the administration of the pharmaceutical composition comprising the AAT RNAi Drug Substance described in Table 2 (ADS-001) to the human subject results in (i) reduction in fibrosis; (ii) reduction in level of periportal hepatocytes; (iii) reduction in serum Z-AAT; (iv) reduction in total liver Z-AAT; (v) reduction in soluble liver Z-AAT; (vi) reduction in insoluble liver Z-AAT; (vii) reduction in ALT; (viii) reduction in GGT; (ix) reduction in Pro-C3; (x) histological improvement in steatosis, or, (xi) a combination thereof.

In some aspects, the reduction in serum Z-AAT is at least about 70%. In some aspects, the reduction in serum Z-AAT is between about 70% and about 100%. In some embodiments, the reduction in serum Z-AAT is about 75%, about 80%, about 85%, about 90%, about 95%, about 97%, or about 99%. In some aspects, the reduction in total liver Z-AAT is at least about 70%. In some aspects, the reduction in total liver Z-AAT is between about 70% and about 100%. In some embodiments, the reduction in total liver Z-AAT is about 75%, about 80%, about 85%, about 90%, about 95%, about 97%, or about 99%. In some aspects, the reduction in soluble liver Z-AAT is at least about 50%. In some aspects, the reduction in soluble liver Z-AAT is between about 50% and about 97%. In some embodiments, the reduction in soluble liver Z-AAT is about 55%, about 60%, about 65%, about 70%, about 80%, about 85%, about 90%, or about 95%. In some aspects, the reduction in insoluble liver Z-AAT is at least about 40%. In some aspects, the reduction in insoluble liver Z-AAT is between about 40% and about 97%. In some embodiments, the reduction in insoluble liver Z-AAT is about 45%, about 50%, about 55%, about 60%, about 65%, about 70%, about 75%, about 80%, about 85%, about 90%, or about 95%. In some aspects, the reduction in ALT is at least about 30%. In some aspects, the reduction in ALT is between about 30% and about 75%. In some embodiments, the reduction in ALT is about 35%, about 40%, about 45%, about 50%, about 55%, about 60%, about 65%, or about 70%. In some aspects, the reduction in GGT is at least about 25%. In some aspects, the reduction in GGT is between about 25% and about 85%. In some embodiments, the reduction in GGT is about 30%, about 35%, about 40%, about 45%, about 50%, about 55%, about 60%, about 65%, about 70%, about 75%, or about 80%. In some aspects, the reduction in fibrosis is at least about 15% as measured by FIBROSCAN®. In some aspects, the reduction in fibrosis is between about 15% and about 90% as measured by FIBROSCAN®. In some embodiments, the reduction in fibrosis is about 20%, about 25%, about 30%, about 35%, about 40%, about 45%, about 50%, about 55%, about 60%, about 65%, about 70%, about 75%, about 80%, or about 85% as measured by FIBROSCAN®. In some aspects, the reduction in Pro-C3 is at least about 15%. In some aspects, the reduction in Pro-C3 is between about 15% and about 90%. In some aspects, the human subject has a histological improvement in steatosis. In some embodiments, the reduction in Pro-C3 is about 20%, about 25%, about 30%, about 35%, about 40%, about 45%, about 50%, about 55%, about 60%, about 65%, about 70%, about 75%, about 80%, or about 85%. In some aspects, the administration of the pharmaceutical composition comprising the AAT RNAi Drug Substance described in Table 2 (ADS-001) to the human subject results in improvements in fibrosis, Portal Inflammation, Interface Hepatitis, Global Portal Tract Involvement, PAS+D Zonal Location, Zone 1 “Globule” Periportal Involvement, or any combination thereof.

All the changes described above are with respect to a predetermined threshold, to the levels in the subject prior to being administered the AAT RNAi Drug Substance, to the levels in a subject not receiving the AAT RNAi Drug Substance, or to control levels determined in a population. Measurements of fibrosis; periportal hepatocytes; serum Z-AAT; liver Z-AAT; soluble liver Z-AAT; insoluble liver Z-AAT; ALT; GGT; Pro-C3; or steatosis are conducted as described in the present disclosure or using methods known in the art.

Other objects, features, aspects, and advantages of the invention will be apparent from the following detailed description, accompanying figures, and from the claims.

### BRIEF DESCRIPTION OF THE DRAWINGS

FIGS. 1A to 1E. Chemical structure representation of AAT RNAi Drug Substance described in Table 2 (referred to herein as ADS-001; i.e., AAT RNAi agent conjugated to a tridentate N-acetyl-galactosamine targeting group at the 5′ terminal end of the sense strand), shown in a sodium salt form.

FIGS. 2A to 2E. Chemical structure representation of AAT RNAi Drug Substance described in Table 2, shown in a free acid form.

FIG. 3. Schematic diagram of the modified sense and antisense strands of AAT RNAi Drug Substance described in Table 2 (referred to herein as ADS-001; i.e., AAT RNAi agent conjugated to a tridentate N-acetyl-galactosamine targeting group at the 5′ terminal end of the sense strand). The following abbreviations are used in FIG. 3: a, c, g, and u are 2′-O-methyl modified nucleotides; Af, Cf, Gf, and Uf are 2′-fluoro (also referred to in the art as 2′-deoxy-2′-fluoro) modified nucleotides; o is a phosphodiester linkage; s is a phosphorothioate linkage; invAb is an inverted abasic residue or subunit; and (NAG37)s is a tridentate N-acetyl-galactosamine targeting ligand having the following chemical structure:

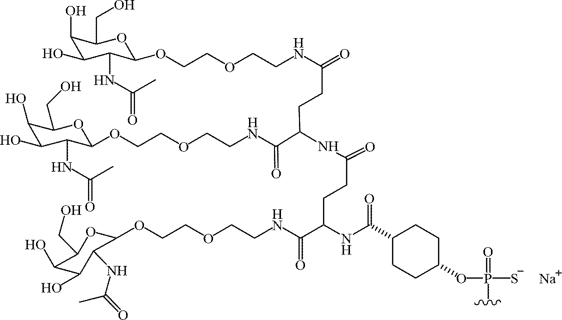

(shown in sodium salt form), or

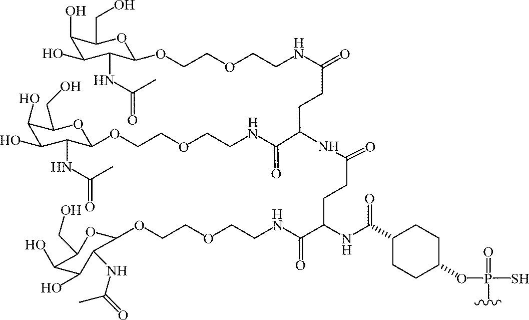

(shown in free acid form).

FIG. 4. Final Phase I study design and dose escalation schedule for the Phase I clinical study described in Example 2.

FIG. 5. Graph showing serum AAT levels in normal health human volunteers (NHV) administered with placebo (all Cohorts) or 35 mg of AAT RNAi Drug Substance (Cohort 1) from the Phase I clinical study described in Example 2. As shown in FIGS. 5 through 11, “Active” refers to the AAT RNAi Drug Substance described in Table 2 (administered as Formulated AAT RNAi Drug Substance as described in Table 3.1).

FIG. 6. Graph showing serum AAT levels in NHVs administered with placebo (all Cohorts) or a single 100 mg dose of AAT RNAi Drug Substance (Cohort 2b) from the Phase I clinical study described in Example 2.

FIG. 7. Graph showing serum AAT levels in NHVs administered with placebo (all Cohorts) or a single 200 mg dose of AAT RNAi Drug Substance (Cohort 3b) from the Phase I clinical study described in Example 2.

FIG. 8. Graph showing serum AAT levels in NHVs administered with placebo (all Cohorts) or a single 300 mg dose of AAT RNAi Drug Substance (Cohort 4b) from the Phase I clinical study described in Example 2.

FIG. 9. Graph showing serum AAT levels in NHVs administered with placebo (all Cohorts) or three 100 mg doses of AAT RNAi Drug Substance administered monthly (Cohort 2) from the Phase I clinical study described in Example 2.

FIG. 10. Graph showing serum AAT levels in NHVs administered with placebo (all Cohorts) or three 200 mg doses of AAT RNAi Drug Substance administered monthly (Cohort 3) from the Phase I clinical study described in Example 2.

FIG. 11. Graph showing serum AAT levels in NHVs administered with placebo (all Cohorts) or three 300 mg doses of AAT RNAi Drug Substance administered monthly (Cohort 4) from the Phase I clinical study described in Example 2.

FIG. 12. Phase II study design and dose escalation schedule for the Phase II clinical study described in Example 3.

FIG. 13. Graph showing serum Z-AAT levels in PiZZ genotype subjects administered with three 200 mg doses of AAT RNAi Drug Substance (Cohort 1) from the Phase II clinical study described in Example 3. Downward pointing arrows on the x-axis indicate the timing of administration. LLOQ: Lower Limit of Quantitation.

FIG. 14. Graph showing serum Z-AAT levels in PiZZ genotype subjects administered with three 100 mg doses of AAT RNAi Drug Substance (Cohort 2) from the Phase II clinical study described in Example 3. Downward pointing arrows on the x-axis indicate the timing of administration. LLOQ: Lower Limit of Quantitation.

### DETAILED DESCRIPTION

### RNAi Agents

The methods described herein include the administration of a pharmaceutical composition to a human subject, wherein the pharmaceutical composition includes a composition that contains an RNAi agent (referred to herein and in the art as an RNAi agent or an RNAi trigger) capable of inhibiting expression of an AAT gene, e.g., ADS-001 or a salt thereof. In some embodiments, the methods described herein include the administration of a pharmaceutical composition to a human subject, wherein the pharmaceutical composition includes the AAT RNAi Drug Substance described in Table 2 (also referred to as ADS-001, or a pharmaceutically acceptable salt thereof). In the context of the present disclosure, the terms “salt thereof” and “pharmaceutically acceptable salt thereof” are considered equivalent and interchangeable. As used herein, the terms “includes” and “comprises” are interchangeable.

The compositions suitable for use in the methods disclosed herein are comprised of an RNAi agent that inhibits expression of an AAT gene in a human subject, and a targeting moiety or targeting group. In some embodiments, the RNAi agent includes the nucleotide sequences provided in Table 1.1 and 1.2, e.g., the antisense oligonucleotide of SEQ ID NO: 2 and the sense oligonucleotide of SEQ ID NO: 4, wherein the sense strand of the RNAi agent, e.g., the sense oligonucleotide of SEQ ID NO: 4, is further linked or conjugated to a targeting group comprising three N-acetyl-galactosamine targeting moieties (see, e.g., Table B). An RNAi agent that inhibits expression of an AAT gene in a human subject is referred to as an “AAT RNAi agent.” The terms “linked” and “conjugated” refer to the covalent attachment between two moieties, e.g., a sense oligonucleotide of SEQ ID NO: 4, and a targeting moiety (e.g., an asialoglycoprotein receptor targeting moiety such as N-acetyl-galactosamine (e.g., NAG37). In some embodiments, “linked” or “conjugated” refers to the attachment of the targeting moiety to an oligonucleotide sequence as a step of the solid phase synthesis process (SPSS), e.g., using a phosphoramidite compound that comprises one or more N-acetyl-galactosamine moieties. In some embodiments, “linked” or “conjugated” refers to the covalent attachment of a sense oligonucleotide of SEQ ID NO: 4, and a targeting moiety (e.g., an asialoglycoprotein receptor targeting moiety such as N-acetyl-galactosamine (e.g., NAG37) as a separate step after SPSS, e.g., by using a bifunctional reagent. As used herein, the terms “linked” and “conjugated” are used interchangeably.

In general, AAT RNAi agents comprise a sense strand (also referred to as a passenger strand) and an antisense strand (also referred to as a guide strand) that are annealed to form a duplex. The AAT RNAi agents disclosed herein include an RNA or RNA-like (e.g., chemically modified RNA) oligonucleotide molecule capable of degrading or inhibiting translation of messenger RNA (mRNA) transcripts of AAT mRNA in a sequence specific manner. The AAT RNAi agents disclosed herein may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that the AAT RNAi agents, as that term is used herein, operate primarily through the RNA interference mechanism, the disclosed RNAi agents are not bound by or limited to any particular pathway or mechanism of action. RNAi agents in general are comprised of a sense strand and an antisense strand that are each 16 to 49 nucleotides in length, and include, but are not limited to: short or small interfering RNAs (siRNAs), double-strand RNAs (dsRNA), micro RNAs (miRNAs), short hairpin RNAs (shRNA), and dicer substrates.

The length of an AAT RNAi agent sense strand is typically 16 to 49 nucleotides in length, and the length of an AAT RNAi agent antisense strand is typically 18 to 49 nucleotides in length. In some embodiments, the sense and antisense strands are independently 17 to 26 nucleotides in length. In some embodiments, the sense and antisense strands are independently 21 to 26 nucleotides in length. In some embodiments, the sense and antisense strands are independently 21 to 24 nucleotides in length. In some embodiments, the sense and/or antisense strands are independently 16, 17, 18, 19, 20, 21, 22, 23, 24, 25, 26, 27, 28, 29, or 30 nucleotides in length. In some embodiments, the sense strand and the antisense strand are both 21 nucleotides in length. The sense and antisense strands can be either the same length or different lengths. The sense and antisense strands can also form overhanging nucleotides on one or both ends of the AAT RNAi agent.

AAT RNAi agents inhibit, silence, or knockdown AAT gene expression. As used herein, the terms “silence,” “reduce,” “inhibit,” “down-regulate,” or “knockdown,” when referring to expression of AAT, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agent as compared to a second cell, group of cells, tissue, organ, or subject that has not or have not been so treated. In some instances, the reduction in gene expression is measured by comparing the baseline levels of AAT mRNA or AAT protein in a human subject prior to administration of a composition that comprises an AAT RNAi agent, with the AAT mRNA or AAT protein levels after administration of the therapeutic.

AAT gene inhibition, silencing, or knockdown may be measured by any appropriate assay or method known in the art. The non-limiting Examples set forth herein, as well as the examples set forth in International Patent Application Publication No. WO 2018/132432, which is incorporated by reference herein in its entirety, provide certain examples of appropriate assays for measuring AAT gene expression inhibition. A reference AAT mRNA gene transcript (SERPINA1) for normal wild type humans (referred to as transcript variant 1; GenBank NM-000295.4) can be found at SEQ ID NO:1.

AAT RNAi agents suitable for use in the methods disclosed herein can be covalently linked or conjugated to a targeting group that includes one or more N-acetyl-galactosamine moieties, e.g., a liver targeting group comprising an asialoglycoprotein receptor targeting moiety such as N-acetyl-galactosamine. In embodiments, AAT RNAi agents suitable for use in the methods disclosed herein are covalently linked or conjugated to a targeting group that includes one or more N-acetyl-galactosamine moieties thereby forming the AAT RNAi Drug Substance described in Table 2, i.e., a duplex RNA (double stranded RNA) comprising a sense strand of SEQ ID NO: 6 and an antisense strand of SEQ ID NO:2.

In some embodiments, the methods described herein include the administration of the AAT RNAi Drug Substance described in Table 2, i.e., a duplex RNA (double stranded RNA) comprising a sense strand of SEQ ID NO: 6 and an antisense strand of SEQ ID NO:2. The AAT RNAi Drug Substance described in Table 2 includes the AAT RNAi agent shown in Table 1.1 (antisense strand of SEQ ID NO: 2) and Table 1.2 (sense strand of SEQ ID NO: 4). The N-acetyl-galactosamine moieties facilitate the targeting of the AAT RNAi agent to the asialoglycoprotein receptors (ASGPr) readily present on the surface of hepatocytes, which leads to internalization of the AAT RNAi agent by endocytosis or other means.

The AAT RNAi agents that can be suitable for use in the methods disclosed herein include an antisense strand that has a region of complementarity to at least a portion of an AAT mRNA, i.e., an AAT mRNA target sequence. AAT RNAi agents and AAT RNAi Drug Substances suitable for use in the disclosed methods are described in International Patent Application Publication No. WO 2018/132432, which as previously noted is incorporated by reference herein in its entirety.

As used herein, the terms “sequence” and “nucleotide sequence” mean a succession or order of nucleobases or nucleotides, described with a succession of letters using standard nomenclature. Unless otherwise indicated, nucleotide sequences are written left to right in 5′ to 3′ orientation. As used herein, the terms “nucleobase” and “nucleotide” have the same meaning as commonly understood in the art. Thus, the term “nucleotide” as used herein, refers to a glycoside comprising a sugar moiety, a base moiety and a covalently linked group (linkage group), such as a phosphate or phosphorothioate internucleoside linkage group, and covers both naturally occurring nucleotides, such as DNA or RNA, and non-naturally occurring nucleotides comprising modified sugar and/or base moieties, which are also referred to as nucleotide analogs herein. Herein, a single nucleotide can be referred to as a monomer or unit.

As used herein, the term “complementary,” when used to describe a first nucleotide sequence (e.g., RNAi agent antisense strand) in relation to a second nucleotide sequence (e.g., RNAi agent sense strand or targeted mRNA sequence), means the ability of an oligonucleotide that includes the first nucleotide sequence to hybridize (form base pair hydrogen bonds under mammalian physiological conditions (or otherwise suitable conditions) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that all (100%) of the bases in a contiguous sequence of a first oligonucleotide will hybridize with the same number of nucleotides in a contiguous sequence of a second oligonucleotide. The contiguous sequence may comprise all or a part of a first or second nucleotide sequence.

As used herein, “partially complementary” means that in a hybridized pair of nucleotide sequences, at least 70%, but not all, of the bases in a contiguous sequence of a first oligonucleotide will hybridize with the same number of bases in a contiguous sequence of a second polynucleotide.

As used herein, “substantially complementary” means that in a hybridized pair of nucleotide sequences, at least 85%, but not all, of the bases in a contiguous sequence of a first oligonucleotide will hybridize with the same number of bases in a contiguous sequence of a second polynucleotide. The terms “complementary,” “fully complementary,” “partially complementary,” and “substantially complementary” herein are used with respect to the nucleotide matching between the sense strand and the antisense strand of an RNAi agent, or between the antisense strand of an RNAi agent and a sequence of an AAT mRNA.

As used herein, the term “substantially identical” or “substantially identity” as applied to nucleic acid sequence means that a nucleic acid sequence comprises a sequence that has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates. In some embodiments, the asymmetric center can be an asymmetric carbon atom.

The terms “identical” or percent “identity” in the context of two or more nucleic acids refer to two or more sequences that are the same or have a specified percentage of nucleotides that are the same, when compared and aligned (introducing gaps, if necessary) for maximum correspondence, not considering any conservative substitutions as part of the sequence identity. The percent identity can be measured using sequence comparison software or algorithms or by visual inspection. Various algorithms and software are known in the art that can be used to obtain alignments of nucleotide sequences.

Sequence alignments can be conducted using methods known in the art such as MAFFT, Clustal (ClustalW, Clustal X or Clustal Omega), MUSCLE, etc.

Different regions within a single polynucleotide target sequence that aligns with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain embodiments, the percentage identity (%ID) or of a first nucleic acid sequence to a second nucleic acid sequence is calculated as %ID = 100 x (Y/Z), where Y is the number of nucleobases scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

### Modified Nucleotides and Modified Internucleoside Linkages

The AAT RNAi agents disclosed herein, e.g., dsRNA targeting an AAT mRNA transcript, can be comprised of modified nucleotides, which can preserve activity of the RNAi agent while at the same time increasing the serum stability, as well as minimize the possibility of activating interferon activity in humans. As used herein, a “modified nucleotide” is a nucleotide other than a ribonucleotide (2′-hydroxyl nucleotide). In some embodiments, at least about 50%, at least about 60%, at least about 70%, at least about 80%, at least about 90%, at least about 95%, at least about 97%, at least about 98%, at least about 99%, or about 100% of the nucleotides are modified nucleotides. In some embodiments, about 50%, about 60%, at about 70%, about 80%, about 90%, about 95%, about 97%, about 98%, about 99%, or about 100% of the nucleotides are modified nucleotides. In some embodiments, between about 50% and about 60%, between about 60% and about 70%, between about 70% and about 80%, between about 80% and about 90%, between about 90% and about 95%, or between about 95% and about 100% of the nucleotides are modified nucleotides. As used herein, modified nucleotides include any known modified nucleotides known in the art, including but not limited to, deoxyribonucleotides, nucleotide mimics, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-arabino nucleotides, 5′-Me, 2′-fluoro nucleotides, morpholino nucleotides, vinyl phosphonate-containing nucleotides, and cyclopropyl phosphonate-containing nucleotides. In some embodiments, the modified nucleotides of an AAT RNAi agent disclosed herein, e.g., ADS-001 or a salt thereof, are 2′-modified nucleotides (i.e. a nucleotide with a group other than a hydroxyl group at the 2′ position of the five-membered sugar ring). 2′-modified nucleotides include, but are not limited to, 2′-O-methyl nucleotides, 2′-deoxy-2′-fluoro nucleotides (commonly referred to simply as 2′-Fluoro nucleotides), 2′-deoxy nucleotides, 2′-methoxyethyl (2′-O-2-methoxyethyl) nucleotides, 2′-amino nucleotides, and 2′-alkyl nucleotides. Additional 2′-modified nucleotides are known in the art. It is not necessary for all nucleotides in a given RNAi agent to be uniformly modified. Additionally, more than one modification can be incorporated in a single AAT RNAi agent or even in a single nucleotide thereof. The AAT RNAi agent sense strands and antisense strands can be synthesized and/or modified by methods known in the art. Modification at one nucleotide is independent of modification at another nucleotide.

In some embodiments, the nucleobase (often referred to as simply the “base”) can be modified. As is commonly used in the art, natural nucleobases include the primary purine bases adenine and guanine, and the primary pyrimidine bases cytosine, thymine, and uracil. A nucleobase may be modified to include, without limitation, universal bases, hydrophobic bases, promiscuous bases, size-expanded bases, and fluorinated bases. See, e.g., Modified Nucleosides in Biochemistry, Biotechnology and Medicine, Herdewijn, P. ed. Wiley-VCH, 2008. The synthesis of such modified nucleobases (including phosphoramidite compounds that include modified nucleobases) is known in the art.

Modified nucleobases include, for example, 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an AAT RNAi agent disclosed herein, e.g., ADS-001 or a salt thereof, are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides.

In some embodiments, one or more nucleotides of an AAT RNAi agent disclosed herein, e.g., ADS-001 or a salt thereof, are linked by non-standard linkages or backbones (i.e., modified internucleoside linkages or modified backbones). Modified internucleoside linkages or backbones include, but are not limited to, phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an AAT RNAi agent disclosed herein, e.g., ADS-001 or a salt thereof, can contain 1, 2, 3, 4, 5, or 6 phosphorothioate linkages, an antisense strand of an AAT RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an AAT RNAi agent disclosed herein, e.g., ADS-001 or a salt thereof, can contain 1, 2, 3, or 4 phosphorothioate linkages, an antisense strand of an AAT RNAi agent disclosed herein, e.g., ADS-001 or a salt thereof, can contain 1, 2, 3, or 4 phosphorothioate linkages, or both the sense strand and the antisense strand independently can contain 1, 2, 3, or 4 phosphorothioate linkages.

In some embodiments, a sense strand of an AAT RNAi agent disclosed herein, e.g., ADS-001 or a salt thereof, contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, phosphorothioate internucleoside linkages are used to link the terminal nucleotides in the sense strand to capping residues present at the 5′-end, the 3′-end, or both the 5′- and 3′-ends of the nucleotide sequence. In some embodiments, phosphorothioate internucleoside linkages are used to link a targeting group to the sense strand.

In some embodiments, an antisense strand of an AAT RNAi agent disclosed herein, e.g., ADS-001 or a salt thereof, contains three or four phosphorothioate internucleoside linkages. In some embodiments, an antisense strand of an AAT RNAi agent disclosed herein, e.g., ADS-001 or a salt thereof, contains three phosphorothioate internucleoside linkages. In some embodiments, the three phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, an AAT RNAi agent disclosed herein, e.g., ADS-001 or a salt thereof, contains at least two phosphorothioate internucleoside linkages in the sense strand and three or four phosphorothioate internucleoside linkages in the antisense strand.

In some embodiments, an AAT RNAi agent disclosed herein, e.g., ADS-001 or a salt thereof, contains one or more modified nucleotides and one or more modified internucleoside linkages. In some embodiments, a 2′-modified nucleoside is combined with modified internucleoside linkage.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table A). (See, e.g., F. Czauderna, Nucleic Acids Res., 2003, 31(11), 2705-16). Capping residues can include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand of an AAT RNAi agent disclosed herein, e.g., ADS-001 or a salt thereof. In some embodiments, the 5′ end and/or the 3′ end of the sense strand of an AAT RNAi agent disclosed herein, e.g., ADS-001 or a salt thereof, may include more than one inverted abasic deoxyribose moiety as a capping residue.

In some embodiments, one or more inverted abasic residues (invAb) are added to the 3′ end of the sense strand of an AAT RNAi agent disclosed herein, e.g., ADS-001 or a salt thereof. In some embodiments, one or more inverted abasic residues (invAb) are added to the 5′ end of the sense strand of an AAT RNAi agent disclosed herein, e.g., ADS-001 or a salt thereof. In some embodiments, one or more inverted abasic residues or inverted abasic sites are inserted between the targeting ligand and the nucleotide sequence of the sense strand of an AAT RNAi agent disclosed herein, e.g., ADS-001 or a salt thereof. In some embodiments, the inclusion of one or more inverted abasic residues or inverted abasic sites at or near the terminal end or terminal ends of the sense strand of an AAT RNAi agent disclosed herein, e.g., ADS-001 or a salt thereof, allows for enhanced activity or other desired properties of the RNAi agent.

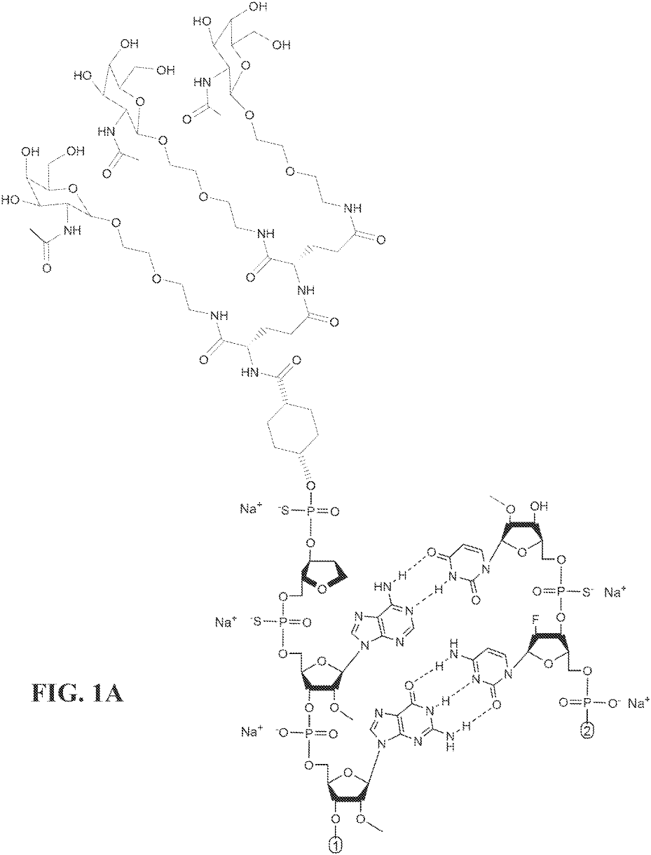

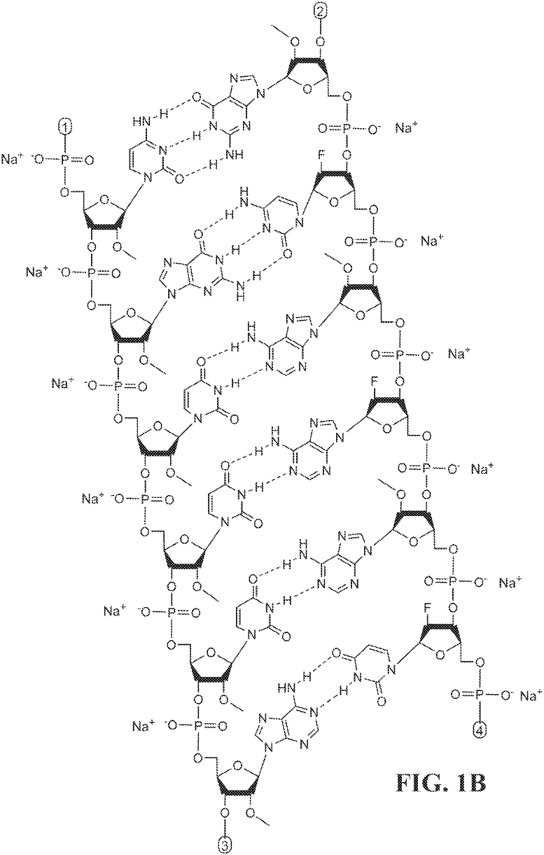

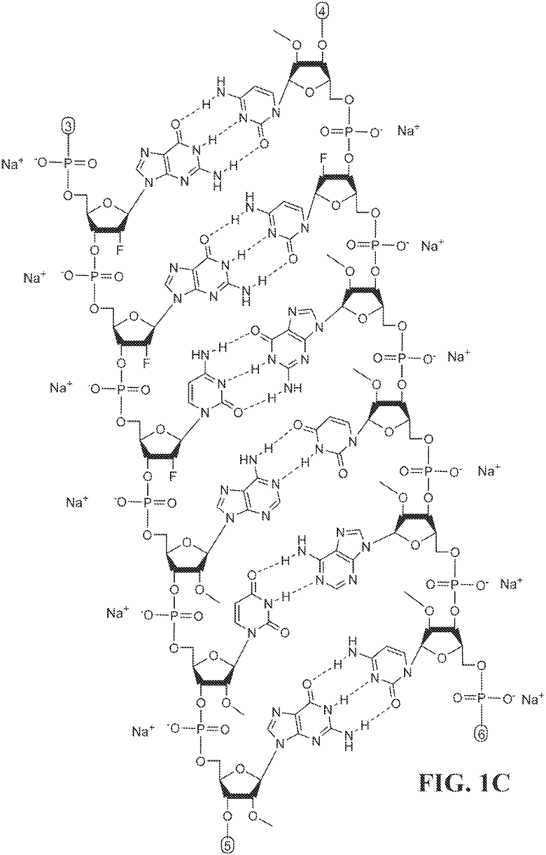

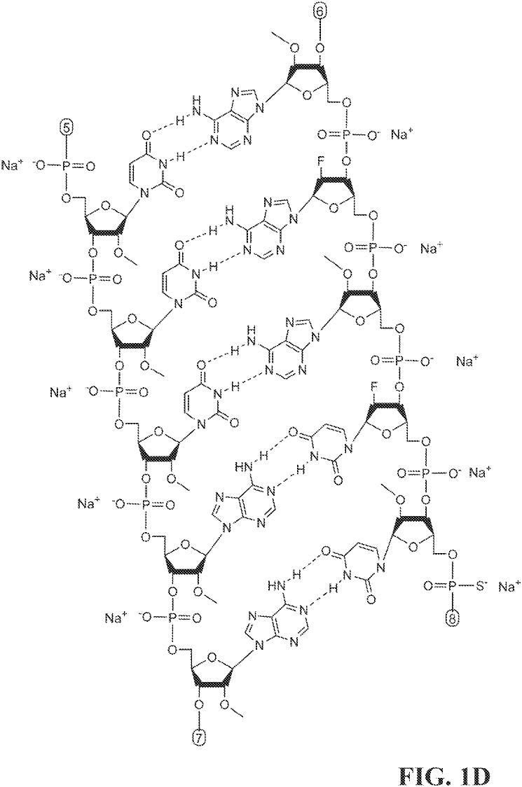

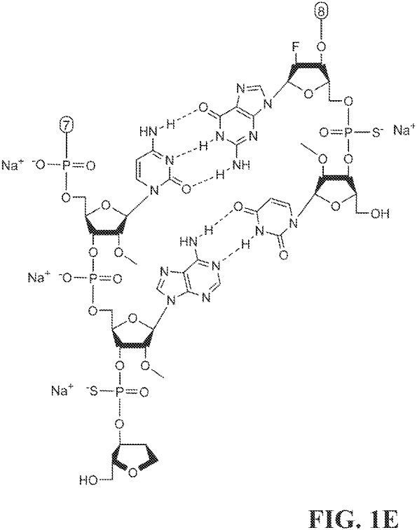

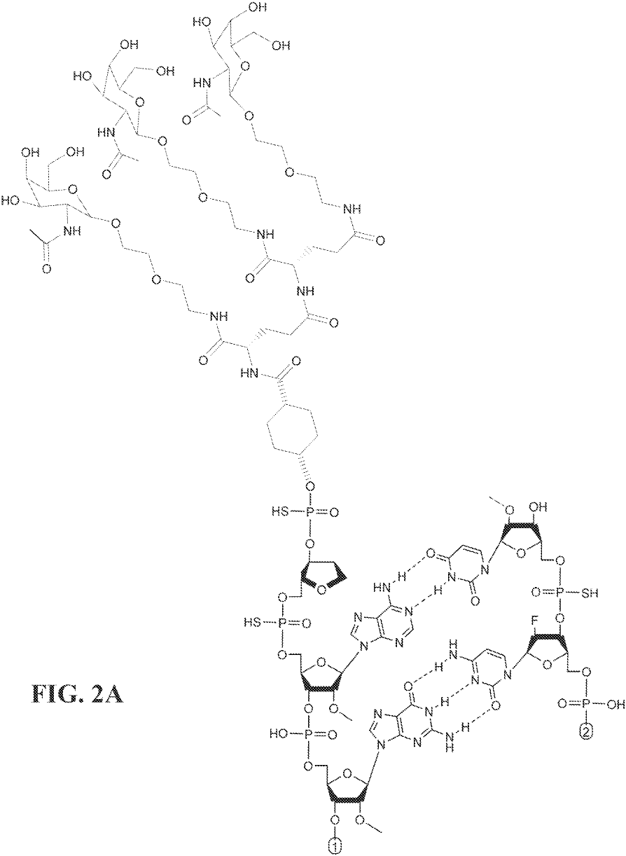

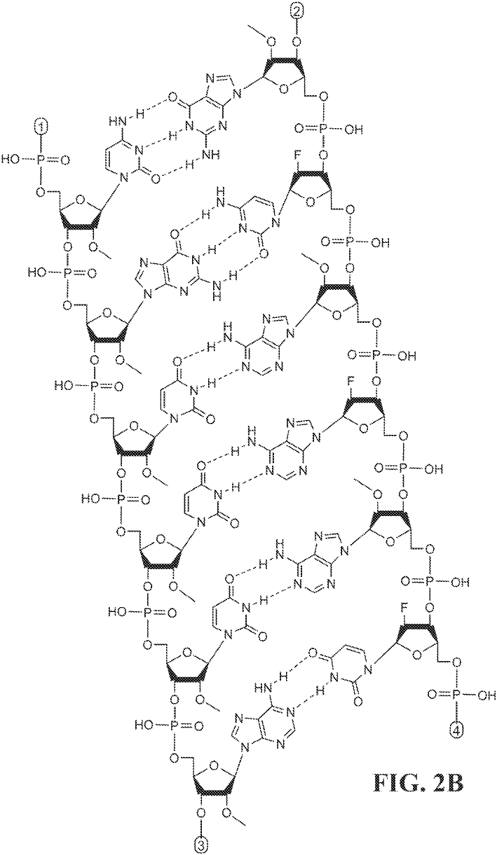

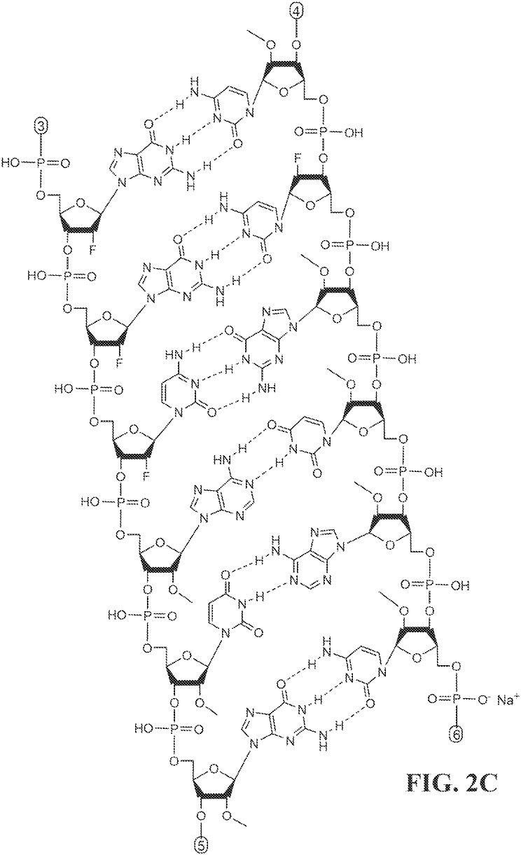

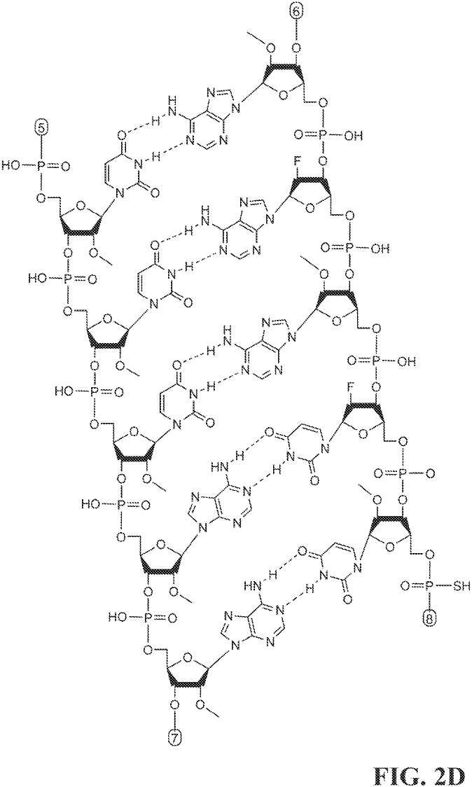

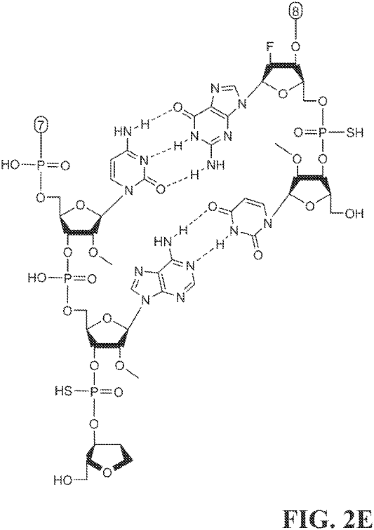

In some embodiments, one or more inverted abasic residues (invAb) are added to the 5′ end of the sense strand of an AAT RNAi agent disclosed herein, e.g., ADS-001 or a salt thereof. In some embodiments, one or more inverted abasic residues can be inserted between the targeting ligand and the nucleotide sequence of the sense strand of an AAT RNAi agent disclosed herein, e.g., ADS-001 or a salt thereof . In some embodiments, the inverted abasic residues may be linked via phosphate, phosphorothioate (e.g., shown herein as (invAb)s)), or other internucleoside linkages. The chemical structures for inverted abasic deoxyribose residues are shown in Table A below, as well as in the chemical structures shown in FIGS. 1A to 1E and FIGS. 2A to 2E.

TABLE A

Inverted Abasic (Deoxyribose) Chemical Structures
When positioned internally on oligonucleotide:

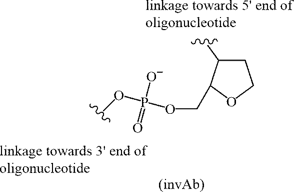

When positioned internally on oligonucleotide:

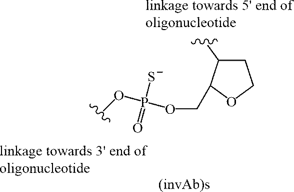

When positioned at the 3′ terminal end of oligonucleotide:

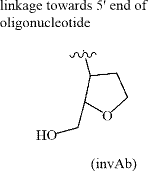

### Targeting Moieties and Groups

An AAT RNAi agent disclosed herein, e.g., ADS-001 or a salt thereof, can comprise an oligonucleotide sequence, e.g., a sense sequence of SEQ ID NO: 4, conjugated to one or more non-nucleotide groups including, but not limited to, a targeting moiety or a targeting group. A targeting moiety or targeting group can enhance targeting or delivery of the RNAi agent. In some embodiments, the targeting moiety or targeting group comprises, e.g., a liver targeting moiety. In some embodiments, the liver targeting moiety can specifically bind to asialoglycoprotein receptor. In some embodiments, the asialoglycoprotein receptor-binding moiety comprises N-acetyl galactosamine (NAG or GalNAc). In some embodiments, the NAG is NAG37. Specific examples of the (NAG37)s targeting group used in the AAT RNAi Drug Substance described in Table 2 herein, which includes three N-acetyl-galactosamine targeting moieties disclosed herein, is provided in Table B. The targeting moiety or targeting group can be covalently linked to the 3′ and/or 5′ end of either the sense strand (e.g., an AAT RNAi agent sense strand of SEQ ID NO: 4) and/or the antisense strand (e.g., an AAT RNAi agent antisense strand of SEQ ID NO: 2). In some embodiments, an AAT RNAi agent contains a targeting group linked to the 3′ and/or 5′ end of the sense strand (e.g., an AAT RNAi agent sense strand of SEQ ID NO: 4). In some embodiments, a targeting group is linked to the 5′ end of an AAT RNAi agent sense strand (e.g., an AAT RNAi agent sense strand of SEQ ID NO: 4). In some embodiments, the targeting group comprises, consists essential of, or consists of the structure (NAG37)s, and is linked to the 5′ end of an AAT RNAi agent sense strand (e.g., an AAT RNAi agent sense strand of SEQ ID NO: 4). A targeting group can be linked directly or indirectly to the RNAi agent via a linker/linking group. In some embodiments, a targeting group is linked to the RNAi agent via a labile, cleavable, or reversible bond or linker. In some embodiments, a targeting group is linked to an inverted abasic residue at the 5′ end of the sense strand.

Targeting groups or targeting moieties can enhance the pharmacokinetic or biodistribution properties of a conjugate or RNAi agent to which they are attached to improve cell-specific distribution and cell-specific uptake of the conjugate or RNAi agent. In some embodiments, a targeting group enhances endocytosis of the RNAi agent. A targeting group can be monovalent, divalent, trivalent, tetravalent, or have higher valency for the target to which it is directed. Representative targeting groups include, without limitation, compounds with affinity to cell surface molecules, cell receptor ligands, haptens, antibodies, monoclonal antibodies, antibody fragments, and antibody mimics with affinity to cell surface molecules.

In some embodiments, a targeting group comprises an asialoglycoprotein receptor ligand. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-acetyl-galactosamine, N-propionyl-galactosamine, N-n-butanoyl-galactosamine, and N-iso-butanoyl-galactosamine (see for example: S.T. Iobst and K. Drickamer, J.B.C., 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative) or multimeric (e.g., having multiple galactose derivatives). The galactose derivative or galactose derivative “cluster” can be attached to the 3′ or 5′ end of the sense or antisense strand of the RNAi agent disclosed herein using methods known in the art.

In some embodiments, a targeting group comprises a galactose derivative cluster. As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamines. In some embodiments, the galactose derivative cluster comprises three N-acetyl-galactosamines. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamines.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, for example, U.S. Pat. No. 5,885,968; Biessen et al. J. Med. Chem. 1995 Vol. 39 p. 1538-1546). The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer.

The preparation of targeting groups, such as galactose derivative clusters that include N-acetyl-galactosamine, is described in, for example, International Patent Application Publication No. WO 2018/044350 (Pat. Application No. PCT/US2017/021147) and International Patent Application Publication No. WO 2017/156012 (Pat. Application No. PCT/US2017/021175), the contents of both of which are incorporated by reference herein in their entirety.

For example, the targeting ligand conjugated to the AAT RNAi agent described in Tables 1.1 and 1.2, i.e., a dsRNA comprising (i) an antisense strand comprising, consisting, or consisting essentially of SEQ ID NO:2 and (ii) a sense strand comprising, consisting, or consisting essentially of SEQ ID NO:4, has the chemical structure of (NAG37)s, as shown in the following Table B.

TABLE B

Chemical Structure of (NAG37)s.

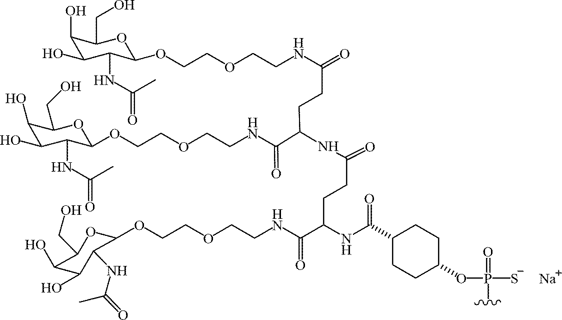

((NAG37)s shown in sodium salt form)

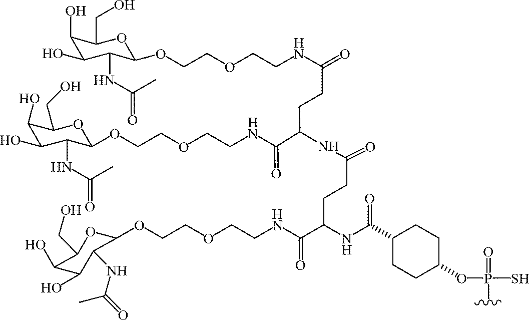

((NAG37) shown in free acid form)

### AAT RNAi Agents and AAT RNAi Drug Substance (ADS-001)

In some embodiments, the AAT RNAi agent used in the methods disclosed herein have the nucleotide sequences of the AAT RNAi Drug Substance (ADS-001) shown in Table 2, or a salt thereof. The nucleotide sequences of the AAT RNAi agent found in AAT RNAi Drug Substance include an antisense strand nucleotide sequence as set forth in SEQ ID NO:2 presented in the following Table 1.1, and a sense strand nucleotide sequence as set forth in SEQ ID NO: 4 presented in the following Table 1.2.

TABLE 1.1

AAT RNAi Agent Antisense Strand Sequence

| SEQ ID NO. | Antisense Sequence (Modified) (5′ → 3′) | SEQ ID NO. | Underlying Base Sequence (5′ → 3′) |
|---|---|---|---|
| 2 | usGfsuUfaAfacaugCfcUfaAfaCfgCfsu | 3 | UGUUAAACAUGCCUAAACGCU |

TABLE 1.2

AAT RNAi Agent Sense Strand Nucleotide Sequence (shown as modified version without inverted abasic residues or NAG targeting group present in AAT RNAi Drug Substance)

| SEQ ID NO. | Sense Sequence (Modified) (5′ → 3′) | SEQ ID NO. | Underlying Base Sequence (5′ → 3′) |
|---|---|---|---|
| 4 | agcguuuaGfGfCfauguuuaaca | 5 | AGCGUUUAGGCAUGUUUAACA |

As used in Tables 1.1, 1.2, and 2 herein, the following notations are used to indicate modified nucleotides, targeting groups, and linking groups: A, G, C, and U represent adenosine, cytidine, guanosine, and uridine, respectively; a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; (invAb) represents an inverted abasic deoxyribose residue (see Table A); and (NAG37)s represents the structure shown in Table B, above.

As the person of ordinary skill in the art would readily understand, unless otherwise indicated by the sequence (such as, for example, by a phosphorothioate linkage “s”), when present in a sense or antisense strand, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′ → 3′, the inverted abasic residues are inserted such that the 3′ position of the deoxyribose is linked at the 3′ end of the preceding monomer on the respective strand. Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the AAT RNAi agents and compositions that include AAT RNAi agents disclosed herein.

Each sense strand and/or antisense strand can have any targeting groups or linking groups listed above, as well as other targeting or linking groups, conjugated to the 5′ and/or 3′ end of the sense and/or antisense strand oligonucleotide sequence.

The antisense strand sequence of an AAT RNAi agent disclosed herein, e,g,, an antisense strand of SEQ ID NO:2, is designed to target AAT mRNA transcripts from both normal and mutant AAT genes, thereby silencing translation of mutant Z-AAT proteins using an RNA interference mechanism for human subjects with AATD.

In some embodiments, the methods disclosed herein use the AAT RNAi Drug Substance set forth in the following Table 2. Thus, in some embodiments, the AAT RNAi Drug Substance comprises a double stranded RNA (dsRNA) comprising a sense strand of SEQ ID NO: 6. In some embodiments, the AAT RNAi Drug Substance comprises a dsRNA comprising an antisense strand of SEQ ID NO: 2. In some embodiments, the AAT RNAi Drug Substance comprises a dsRNA comprising a sense strand of SEQ ID NO: 6 and an antisense strand of SEQ ID NO: 2. Thus, in some embodiments, the AAT RNAi Drug Substance comprises a double stranded RNA (dsRNA) comprising a sense strand consisting of SEQ ID NO: 6. In some embodiments, the AAT RNAi Drug Substance comprises a dsRNA comprising an antisense strand consisting of SEQ ID NO: 2. In some embodiments, the AAT RNAi Drug Substance comprises a dsRNA comprising a sense strand consisting of SEQ ID NO: 6 and an antisense strand consisting of SEQ ID NO: 2.

TABLE 2

AAT RNAi Drug Substance (ADS-001)
Sense and Antisense Strands (The sense and antisense strands are annealed to form a duplex):
Sense Strand (Modified Sequence) (5′ → 3′): (NAG37)s(invAb)sagcguuuaGfGfCfauguuuaacas(invAb) (SEQ ID NO:6)
Antisense Strand (Modified Sequence) (5′ → 3′): usGfsuUfaAfacaugCfcUfaAfaCfgCfsu (SEQ ID NO:2)

TABLE 2.1

Properties of AAT RNAi Drug Substance (ADS-001) Described in Table 2

| | |
|---|---|
| Chemical Formula: | C₄₉₀H_{61O}F₁₁N₁₆₃Na₄₃O₃₁₂P₄₃S₆ (Na+ form) |
| | C₄₉₃H₆₅₃F₁₁N₁₆₃O₃₁₂P₄₃S₆ (H+ form) |
| Molecular Weight: | 16532.9 Da (Na+ form) |
| | 15587.6 Da (H+ form) |
| Physical Appearance: | White to Off-white Powder |

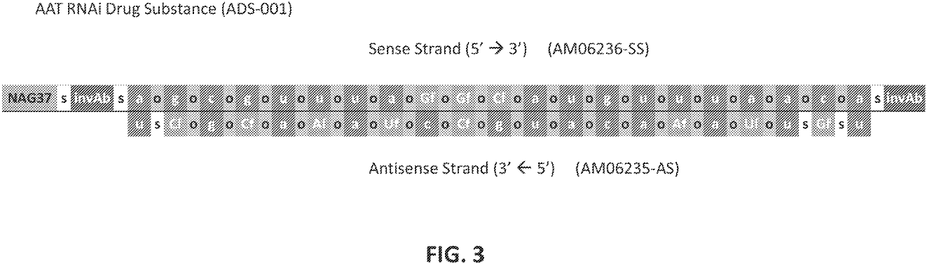

A schematic representation of AAT RNAi Drug Substance (ADS-001) is shown in FIG. 3, and full chemical structure representations are shown in FIGS. 1A to 1E (sodium salt form) and FIGS. 2A to 2E (free acid form). In some embodiments, the AAT RNAi Drug Substance (e.g., ADS-001) is prepared or provided as a salt, mixed salt, or a free-acid. In some embodiments, the AAT RNAi Drug Substance (e.g., ADS-001) is prepared or provided as a sodium salt.

### Pharmaceutical Compositions and Formulations

The AAT RNAi agents suitable for use in the methods disclosed herein, e.g., ADS-001 or a salt thereof, can be prepared as pharmaceutical compositions or formulations for administration to human subjects. The pharmaceutical compositions can be used to treat a subject having a disease or disorder that would benefit from inhibition of expression of AAT mRNA or reduction in the level of AAT protein, such as human subjects having AATD. In some embodiments, the methods include administering an AAT RNAi agent that is linked to a targeting group or targeting ligand as described herein, e.g., a liver-targeting NAG moiety, to a subject in need of treatment. In some embodiments, one or more pharmaceutically acceptable excipients (including vehicles, carriers, diluents, and/or delivery polymers) are added to the pharmaceutical compositions that include an AAT RNAi agent disclosed herein, e.g., ADS-001 or a salt thereof, thereby forming a pharmaceutical formulation suitable for in vivo delivery to a human subject.

The pharmaceutical compositions that include an AAT RNAi agent disclosed herein, e.g., ADS-001 or a salt thereof, when administered to a human subject using the methods disclosed herein, decrease the level of AAT mRNA in the subject.

In some embodiments, the described pharmaceutical compositions including an AAT RNAi agent disclosed herein, e.g., ADS-001 or a salt thereof, are used for treating or managing clinical presentations in a subject with AATD, such as chronic hepatitis, cirrhosis, increased risk of hepatocellular carcinoma, transaminitis, cholestasis, fibrosis, and even fulminant hepatic failure. In some embodiments, a therapeutically or prophylactically effective amount of one or more of pharmaceutical compositions including an AAT RNAi agent disclosed herein, e.g., ADS-001 or a salt thereof, is administered to a subject in need of such treatment. In some embodiments, administration of an AAT RNAi agent disclosed herein, e.g., ADS-001 or a salt thereof, can be used to decrease the number, severity, and/or frequency of symptoms of a disease in a subject.

“Effective amount,” as used herein, is intended to include the amount of an agent or composition that, when administered to a patient for treating a subject having a AATD, is sufficient to effect treatment of the disease (e.g., by diminishing, ameliorating, or maintaining the existing disease or one or more symptoms of disease or its related comorbidities). The “effective amount” may vary depending on the agent or composition, how it is administered, the disease and its severity and the history, age, weight, family history, genetic makeup, stage of pathological processes mediated by AATD, the types of preceding or concomitant treatments, if any, and other individual characteristics of the patient to be treated.

The described pharmaceutical compositions that include an AAT RNAi agent disclosed herein, e.g., ADS-001 or a salt thereof, can be used to treat at least one symptom in a subject having a disease or disorder that would benefit from reduction or inhibition in expression of AAT mRNA. In some embodiments, the subject is administered a therapeutically effective amount of one or more pharmaceutical compositions including an AAT RNAi agent disclosed herein, e.g., ADS-001 or a salt thereof, thereby treating the symptom. In other embodiments, the subject is administered a prophylactically effective amount of one or more AAT RNAi agent disclosed herein, e.g., ADS-001 or a salt thereof, thereby preventing the at least one symptom.

The AAT RNAi agents disclosed herein, e.g., ADS-001 or a salt thereof, can be administered via any suitable route in a preparation appropriately tailored to the particular route. Thus, herein described pharmaceutical compositions can be administered by injection, for example, intravenously or subcutaneously. In some embodiments, the herein described pharmaceutical compositions are administered via subcutaneous injection.

As used herein, a pharmaceutical composition or medicament includes a pharmacologically effective amount of at least one AAT RNAi agent disclosed herein, e.g., ADS-001 or a salt thereof, and one or more pharmaceutically acceptable excipients. Pharmaceutically acceptable excipients (excipients) are substances other than the Active Pharmaceutical Ingredient (API, therapeutic product, e.g., AAT RNAi agent) that are intentionally included in the drug delivery system. Excipients do not exert or are not intended to exert a therapeutic effect at the intended dosage. Excipients can act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients may include, but are not limited to: absorption enhancers, anti-adherents, antifoaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble). For subcutaneous or intravenous administration, suitable carriers may include physiological saline, bacteriostatic water, CREMOPHOR® ELTM (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above.

In some embodiments, a pharmaceutical composition suitable for use in the methods disclosed herein includes the components identified in the Formulated AAT RNAi Drug Substance provided in Table 3.1 or Table 3.2, below.

The AAT RNAi agents disclosed herein, e.g., ADS-001 or a salt thereof, can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier.

In some embodiments, the dosage unit is between about 5 mg and about 300 mg of AAT RNAi Drug Substance, e.g., the Formulated AAT RNAi Drug Substance provided in Table 3.1 or Table 3.2. In some embodiments, the dosage unit is between about 25 mg and about 200 mg of AAT RNAi Drug Substance, e.g., the Formulated AAT RNAi Drug Substance provided in Table 3.1 or Table 3.2. In some embodiments, the dosage unit is between about 100 mg and about 200 mg of AAT RNAi Drug Substance, e.g., the Formulated AAT RNAi Drug Substance provided in Table 3.1 or Table 3.2. In some embodiments, the dosage unit is about 100 mg of AAT RNAi Drug Substance, e.g., the Formulated AAT RNAi Drug Substance provided in Table 3.1 or Table 3.2. In some embodiments, the dosage unit is about 200 mg of AAT RNAi Drug Substance, e.g., the Formulated AAT RNAi Drug Substance provided in Table 3.1 or Table 3.2. In some embodiments, the dosage unit is at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg of AAT RNAi Drug Substance, e.g., the Formulated AAT RNAi Drug Substance provided in Table 3.1 or Table 3.2. In some embodiments, the dosage unit is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, or about 200 mg of AAT RNAi Drug Substance, e.g., the Formulated AAT RNAi Drug Substance provided in Table 3.1 or Table 3.2. In some embodiments, the dosage unit is between about 5 and about 10 mg, between about 10 and about 15 mg, between about 15 and about 20 mg, between about 20 and about 25 mg, between about 25 and about 30 mg, between about 30 and about 35 mg, between about 35 and about 40 mg, between about 40 and about 45 mg, between about 45 and about 50 mg, between about 50 and about 55 mg, between about 55 and about 60 mg, between about 60 and about 65 mg, between about 65 and about 70 mg, between about 70 and about 75 mg, between about 75 and about 80 mg, between about 80 and about 85 mg, between about 85 and about 90 mg, between about 90 and about 95 mg, between about 95 and about 100 mg, between about 100 and about 110 mg, between about 110 and about 120 mg, between about 120 and about 130 mg, between about 130 and about 140 mg, between about 140 and about 150 mg, between about 150 and about 160 mg, between about 160 and about 170 mg, between about 170 and about 180 mg, between about 180 and about 190 mg, or between about 190 and about 200 mg of AAT RNAi Drug Substance, e.g., the Formulated AAT RNAi Drug Substance provided in Table 3.1 or Table 3.2.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.).

As used herein, “pharmacologically effective amount,” “therapeutically effective amount,” or simply “effective amount” refers to that amount of an RNAi agent to produce a pharmacological, therapeutic or preventive result.

The described pharmaceutically acceptable formulations can be packaged into kits, containers, packs, or dispensers. The pharmaceutical compositions described herein can be packaged in pre-filled syringes or vials.

### Formulated AAT RNAi Drug Substance

In some embodiments, the AAT RNAi Drug Substance as provided in Table 2 (e.g., ADS-001 or a salt thereof) is formulated with one or more pharmaceutically acceptable excipients to form a pharmaceutical composition suitable for administration to a human subject.

In some embodiments, the AAT RNAi Drug Substance described in Table 2 is formulated at 230 mg/mL in an aqueous sodium phosphate buffer (0.5 mM sodium phosphate monobasic, 0.5 mM sodium phosphate dibasic), forming the Formulated AAT RNAi Drug Substance (ADS-001-1) shown in Table 3.1:

TABLE 3.1

Composition of Formulated AAT RNAi Drug Substance, per 1.0 mL

| | | | |
|---|---|---|---|
| Component | Function | Quality / Grade | Concentration |
| ADS-001 | Active ingredient | In-house | 230 mg |
| Sodium phosphate monobasic, monohydrate | Suspending agent | USP, Ph. Eur | 0.061 mg |
| Sodium phosphate dibasic, anhydrous | Suspending agent | USP, Ph. Eur | 0.062 mg |
| Water for injection (WFI) | Vehicle | USP, Ph. Eur | 879.2 mg |

In some embodiments, the AAT RNAi Drug Substance described in Table 2 is formulated at 200 mg/mL in an aqueous sodium phosphate buffer (0.5 mM sodium phosphate monobasic, 0.5 mM sodium phosphate dibasic), forming the Formulated AAT RNAi Drug Substance (ADS-001-2) shown in Table 3.2:

TABLE 3.2

Composition of Formulated AAT RNAi Drug Substance, per 1.0 mL

| | | | |
|---|---|---|---|
| Component | Function | Quality / Grade | Concentration |
| ADS-001 | Active ingredient | In-house | 200 mg |
| Sodium phosphate monobasic, monohydrate | Suspending agent | USP, Ph. Eur | ~0.061 mg |
| Sodium phosphate dibasic, anhydrous | Suspending agent | USP, Ph. Eur | ~0.063 mg |
| Water for injection (WFI) | Vehicle | USP, Ph. Eur | ~891 mg |

In some embodiments, a Formulated AAT RNAi Drug Substance of the present disclosure comprises between 150 mg and 250 mg of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof per 1 mL. In some embodiments, a Formulated AAT RNAi Drug Substance of the present disclosure comprises at least about 150 mg, at least about 160 mg, at least about 170 mg, at least about 180 mg, at least about 190 mg, at least about 200 mg, at least about 210 mg, at least about 220 mg, at least about 230 mg, at least about 240 mg, or at least about 250 mg of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof per 1 mL. In some embodiments, a Formulated AAT RNAi Drug Substance of the present disclosure comprises about 150 mg, about 160 mg, about 170 mg, about 180 mg, about 190 mg, about 200 mg, about 210 mg, about 220 mg, about 230 mg, about 240 mg, or about 250 mg of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof per 1 mL. In some embodiments, a Formulated AAT RNAi Drug Substance of the present disclosure comprises between about 150 mg and about 160 mg, about 160 mg and about 170 mg, about 170 mg and about 180 mg, about 180 mg and about 190 mg, about 190 mg and about 200 mg, about 200 mg and about 210 mg, about 210 mg and about 220 mg, about 220 mg and about 230 mg, about 230 mg and about 240 mg, or about 240 mg and about 250 mg of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof per 1 mL.

In some embodiments, a Formulated AAT RNAi Drug Substance of the present disclosure comprises about 0.120 mg of suspending agent per 1 mL. In some embodiments, the suspending agent comprises a phosphate salt or a combination thereof. In some embodiments, the suspending agent comprises a sodium phosphate salt or a combination thereof. In some embodiments, the suspending agent comprises monobasic sodium phosphate. In some embodiments, the suspending agent comprises dibasic sodium phosphate. In some embodiments, the suspending agent comprises monobasic sodium phosphate and dibasic sodium phosphate. In some embodiments, the monobasic sodium phosphate is monohydrate monobasic sodium phosphate. In some embodiments, the dibasic sodium phosphate is anhydrous dibasic sodium phosphate. In some embodiments, the Formulated AAT RNAi Drug Substance of the present disclosure comprises approximately equivalent amounts of monohydrate monobasic sodium phosphate and anhydrous dibasic sodium phosphate. In some embodiments, the Formulated AAT RNAi Drug Substance of the present disclosure comprises approximately 0.061 mg of monohydrate monobasic sodium phosphate per 1 mL. In some embodiments, the Formulated AAT RNAi Drug Substance of the present disclosure comprises approximately 0.062 mg anhydrous dibasic sodium phosphate per 1 mL. In some embodiments, the Formulated AAT RNAi Drug Substance of the present disclosure comprises approximately 0.061 mg of monohydrate monobasic sodium phosphate and approximately 0.062 mg anhydrous dibasic sodium phosphate per 1 mL.

The Formulated AAT RNAi Drug Substance according to Table 3.1 and Table 3.2 is prepared as a sterile formulation. In some embodiments, the Formulated AAT RNAi Drug Substance is packaged in a container, such as a glass vial. In some embodiments, the Formulated AAT RNAi Drug Substance is packaged in a glass vial with a fill volume of about 1.1 mL, and a desired volume for administration can be calculated based upon the desired dose level to be administered.

In some embodiments, the Formulated AAT RNAi Drug Substance set forth in Table 3.1 and Table 3.2 is administered to a human subject using the methods disclosed herein.

### Kits

Any of the compositions described herein, e.g., AAT RNAi agent, AAT RNAi Drug Substance (ADS-001), or a salt thereof, the pharmaceutical compositions and formulations of the AAT RNAi agent or the AAT RNAi Drug Substance (ADS-001), of a salt thereof, or the Formulated AAT RNAi Drug Substance (ADS-001) may be comprised in a kit. In a non-limiting example, the kit comprises an AAT RNAi Drug Substance (ADS-001), or a salt thereof.

The kit may further include reagents or instructions for using the composition described herein in a subject. It may also include one or more buffers.

In some embodiments, the kit may further include an effective amount of additional therapeutic for the treatment of AATD.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the compositions of the invention, e.g., the AAT RNAi Drug Substance (ADS-001), or a salt thereof, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### Pre-Filled Syringes

Any of the compositions described herein, e.g., AAT RNAi agent, AAT RNAi Drug Substance (ADS-001), or a salt thereof, the pharmaceutical compositions and formulations of the AAT RNAi agent or the AAT RNAi Drug Substance (ADS-001), or a salt thereof, or the Formulated AAT RNAi Drug Substance (ADS-001) may be packaged in a syringe. In a non-limiting example, the pre-filled syringe comprises an AAT RNAi Drug Substance (ADS-001), or a salt thereof. In some embodiment, the pre-filled syringe comprises an AAT RNAi Drug Substance (ADS-001) in a dosage unit, e.g., about 100 mg or about 200 mg.

### Human Subjects With AATD and AATD Diagnosis

The methods disclosed herein include treating alpha-1 antitrypsin deficiency (AATD) in a human subject in need thereof, including treatment of the symptoms and diseases caused by AATD in the human subject, using an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, for example, a pharmaceutical composition that comprises the AAT RNAi Drug Substance described in Table 2. In some embodiments, the pharmaceutical compositions comprises a Formulated AAT RNAi Drug Substances set forth in Table 3.1 or Table 3.2.

In some embodiments, the human subject is diagnosed with AATD prior to administration. As noted herein, AATD is a genetic disorder caused by mutations in the gene transcript that results in translation of a mutant form of AAT protein, for which some mutant forms which are prone to abnormal folding lead to intracellular retention in hepatocytes. While various mutations of the SERPINA1 gene have been identified, the most common and serious form of AATD, the PiZZ genotype, is caused by a single base-pair substitution. In subjects with the PiZZ genotype, circulating AAT levels are often reported as less than 15% of levels in normal humans. In many cases, subjects are initially diagnosed with COPD, asthma, or other lung disease without identification of the underlying cause. Over time, liver disease such as fibrosis and cirrhosis can develop due to the intercellular retention of the misfolded (“Z-AAT”) protein and the inability to properly secrete the protein from liver cells. Pediatric subjects typically present with clinical symptoms of liver disease, which may include asymptomatic chronic hepatitis, failure to thrive, poor feeding, or hepatomegaly and splenomegaly. AATD can be diagnosed and confirmed through standard genotyping of blood samples from the subject.

### Dosing and Inhibition of AAT Gene Expression

Generally, an effective amount of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, administered to a subject in need thereof will be in the range of from about 0.1 mg/kg to about 10 mg/kg of body weight/dose, e.g., from about 0.25 mg/kg to about 5 mg/kg of body weight/dose.

In some embodiments, an effective amount of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, will be in the range of from about 0.5 mg/kg to about 4 mg/kg of body weight per dose.

In some embodiments, an effective amount of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is at least about 0.2 mg/kg, at least about 0.4 mg/kg, at least about 0.6 mg/kg, at least about 0.8 mg/kg, at least about 1 mg/kg, at least about 1.2 mg/kg, at least about 1.4 mg/kg, at least about 1.6 mg/kg, at least about 1.8 mg/kg, at least about 2 mg/kg, at least about 2.2 mg/kg, at least about 2.4 mg/kg, at least about 2.6 mg/kg, at least about 2.8 mg/kg, at least about 3 mg/kg, at least about 3.2 mg/kg, at least about 3.4 mg/kg, at least about 3.6 mg/kg, at least about 3.8 mg/kg, at least about 4 mg/kg, at least about 4.2 mg/kg, at least about 4.4 mg/kg, at least about 4.6 mg/kg, at least about 4.8 mg/kg, at least about 5 mg/kg, at least about 5.2 mg/kg, at least about 5.4 mg/kg, at least about 5.6 mg/kg, at least about 5.8 mg/kg, at least about 6 mg/kg, at least about 6.2 mg/kg, at least about 6.4 mg/kg, at least about 6.6 mg/kg, at least about 6.8 mg/kg, at least about 7 mg/kg, at least about 7.2 mg/kg, at least about 7.4 mg/kg, at least about 7.6 mg/kg, at least about 7.8 mg/kg, at least about 8 mg/kg, at least about 8.2 mg/kg, at least about 8.4 mg/kg, at least about 8.6 mg/kg, at least about 8.8 mg/kg, at least about 9 mg/kg, at least about 9.2 mg/kg, at least about 9.4 mg/kg, at least about 9.6 mg/kg, at least about 9.8 mg/kg, at least about 10 mg/kg, of body weight per dose.

In some embodiments, an effective amount of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is about 0.2 mg/kg, about 0.4 mg/kg, about 0.6 mg/kg, about 0.8 mg/kg, about 1 mg/kg, about 1.2 mg/kg, about 1.4 mg/kg, about 1.6 mg/kg, about 1.8 mg/kg, about 2 mg/kg, about 2.2 mg/kg, about 2.4 mg/kg, about 2.6 mg/kg, about 2.8 mg/kg, about 3 mg/kg, about 3.2 mg/kg, about 3.4 mg/kg, about 3.6 mg/kg, about 3.8 mg/kg, about 4 mg/kg, about 4.2 mg/kg, about 4.4 mg/kg, about 4.6 mg/kg, about 4.8 mg/kg, about 5 mg/kg, about 5.2 mg/kg, about 5.4 mg/kg, about 5.6 mg/kg, about 5.8 mg/kg, about 6 mg/kg, about 6.2 mg/kg, about 6.4 mg/kg, about 6.6 mg/kg, about 6.8 mg/kg, about 7 mg/kg, about 7.2 mg/kg, about 7.4 mg/kg, about 7.6 mg/kg, about 7.8 mg/kg, about 8 mg/kg, about 8.2 mg/kg, about 8.4 mg/kg, about 8.6 mg/kg, about 8.8 mg/kg, about 9 mg/kg, about 9.2 mg/kg, about 9.4 mg/kg, about 9.6 mg/kg, about 9.8 mg/kg, about 10 mg/kg, of body weight per dose.

In some embodiments, an effective amount of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is between about 1 mg/kg and about 2 mg/kg, about 2 mg/kg and about 3 mg/kg, between about 3 mg/kg and about 4 mg/kg, between about 4 mg/kg and about 5 mg/kg, between about 5 mg/kg and about 6 mg/kg, between about 6 mg/kg and about 7 mg/kg, between about 7 mg/kg and about 8 mg/kg, between about 8 mg/kg and about 9 mg/kg, and between about 9 mg/kg and about 10 mg/kg of body weight per dose.

In some embodiments, the effective amount is a fixed dose. In some embodiments, a fixed dose of between 5 mg to 300 mg of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is an effective dose. In some embodiments, a fixed dose of between 25 mg to 200 mg of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is an effective dose. The amount administered will likely depend on such variables as the overall age and health status of the subject, the relative biological efficacy of the compound delivered, the formulation of the drug, the presence and types of excipients in the formulation, and the route of administration. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of from about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 140 mg, about 160 mg, about 180 mg, about 200 mg, about 220 mg, about 240 mg, about 260 mg or about 280 mg to about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 120 mg, about 140 mg, about 160 mg, about 180 mg, about 200 mg, about 220 mg, about 240 mg, about 260 mg, about 280 mg or about 300 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 25 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 50 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 75 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 100 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 125 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 150 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 175 mg is an effective dose. In some embodiments, a fixed dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, of about 200 mg is an effective dose.

Also, it is to be understood that the initial dosage administered can, in some instances, be increased beyond the above upper level to rapidly achieve the desired blood-level or tissue level, or the initial dosage can, in some instances, be smaller than the optimum. For example, in some embodiments, an initial dose of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, from about 25 mg to about 200 mg, e.g., about 25 mg, about 50 mg, about 75 mg, about 100 mg, about 125 mg, about 150 mg, about 175 mg, or about 200 mg, is administered, followed by a second dose of from about 25 to 200 mg approximately four weeks or 1 month later, and thereafter additional doses (a concept similar to “maintenance doses”) are administered once about every twelve weeks or about every three months (i.e., about once per quarter).

For treatment of disease or for formation of a medicament or composition for treatment of a disease, the pharmaceutical compositions described herein including an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, can be combined with an excipient or with a second therapeutic agent or treatment including, but not limited to: a second or other RNAi agent, a small molecule drug, an antibody, an antibody fragment, peptide and/or aptamer.

In some aspects of the methods disclosed herein, the administration of a pharmaceutical composition including an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, to a human subject in need thereof can result in (i) reduction in fibrosis; (ii) reduction in level of periportal hepatocytes; (iii) reduction in serum Z-AAT; (iv) reduction in total liver Z-AAT; (v) reduction in soluble liver Z-AAT; (vi) reduction in insoluble liver Z-AAT; (vii) reduction in ALT; (viii) reduction in GGT; (ix) reduction in Pro-C3; or, (x) a combination thereof.

In some embodiments, the administration of a pharmaceutical composition including an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, to a human subject in need thereof can result in improvements in fibrosis, Portal Inflammation, Interface Hepatitis, Global Portal Tract Involvement, PAS+D Zonal Location, Zone 1 “Globule” Periportal Involvement, or any combination thereof.

In some embodiments, the gene expression level and/or mRNA level of an AAT gene in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 96%, at least about 97%, at least about 98%, at least about 99%, or greater than 99% relative to the subject prior to being administered the AAT RNAi Drug Substance or to a subject not receiving the AAT RNAi Drug Substance. The gene expression level and/or mRNA level in the subject is reduced in a cell, group of cells, and/or tissue of the subject. In some embodiments, the gene expression level and/or mRNA level in the subject is reduced in a liver cell, e.g., a hepatocyte, a hepatic stellate cell, group of liver cells, and/or liver of the subject.

In some embodiments, the gene expression level and/or mRNA level of an AAT gene in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is administered is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 95%, about 96%, about 97%, about 98%, about 99%, or greater than 99% relative to the subject prior to being administered the AAT RNAi Drug Substance or to a subject not receiving the AAT RNAi Drug Substance.

In some embodiments, the gene expression level and/or mRNA level of an AAT gene in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, is administered is reduced by between about 5% and about 10%, between about 10% and about 15%, between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, or between about 95% and 100% relative to the subject prior to being administered the AAT RNAi Drug Substance or to a subject not receiving the AAT RNAi Drug Substance.

In some embodiments, the protein level of AAT in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater than about 99% relative to the subject prior to being administered the AAT RNAi Drug Substance or to a subject not receiving the AAT RNAi Drug Substance. The protein level in the subject is reduced in a cell, group of cells, tissue, blood, and/or other fluid of the subject. In some embodiments, the protein level in the subject is reduced in a liver cell, e.g., a hepatocyte, a hepatic stellate cell, group of liver cells, and/or liver of the subject.

In some embodiments, the protein level of AAT in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or greater than about 99% relative to the subject prior to being administered the AAT RNAi Drug Substance or to a subject not receiving the AAT RNAi Drug Substance.

In some embodiments, the protein level of AAT in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by between about 5% and about 10%, between about 10% and about 15%, between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, or between about 95% and 100% relative to the subject prior to being administered the AAT RNAi Drug Substance or to a subject not receiving the AAT RNAi Drug Substance.

In some embodiments, the liver Z-AAT protein level in a subject having AATD to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater than 99% relative to the subject prior to being administered the AAT RNAi Drug Substance or to a subject not receiving the AAT RNAi Drug Substance.

In some embodiments, the liver Z-AAT protein level in a subject having AATD to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or greater than 99% relative to the subject prior to being administered the AAT RNAi Drug Substance or to a subject not receiving the AAT RNAi Drug Substance.

In some embodiments, the liver Z-AAT protein level in a subject having AATD to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by between about 5% and about 10%, between about 10% and about 15%, between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, or between about 95% and 100% relative to the subject prior to being administered the AAT RNAi Drug Substance or to a subject not receiving the AAT RNAi Drug Substance.

In some embodiments, the liver Z-AAT soluble or monomer protein level in a subject having AATD to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater than about 99% relative to the subject prior to being administered the AAT RNAi Drug Substance or to a subject not receiving the AAT RNAi Drug Substance.

In some embodiments, the liver Z-AAT soluble or monomer protein level in a subject having AATD to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or greater than about 99% relative to the subject prior to being administered the AAT RNAi Drug Substance or to a subject not receiving the AAT RNAi Drug Substance.

In some embodiments, the liver Z-AAT soluble or monomer protein level in a subject having AATD to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by between about 5% and about 10%, between about 10% and about 15%, between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, or between about 95% and 100% relative to the subject prior to being administered the AAT RNAi Drug Substance or to a subject not receiving the AAT RNAi Drug Substance.

In some embodiments, the reduction in soluble liver Z-AAT after administering ADS-001 or a salt thereof to a subject in need thereof is at least about 50%. In some embodiments, the reduction in soluble liver Z-AAT after administering ADS-001 or a salt thereof to a subject in need thereof is about 50%. In some embodiments, the reduction in soluble liver Z-AAT after administering ADS-001 or a salt thereof to a subject in need thereof is at least about 55%. In some embodiments, the reduction in soluble liver Z-AAT after administering ADS-001 or a salt thereof to a subject in need thereof is about 55%. In some embodiments, the reduction in soluble liver Z-AAT after administering ADS-001 or a salt thereof to a subject in need thereof is at least about 60%. In some embodiments, the reduction in soluble liver Z-AAT after administering ADS-001 or a salt thereof to a subject in need thereof is about 60%. In some embodiments, the reduction in soluble liver Z-AAT after administering ADS-001 or a salt thereof to a subject in need thereof is at least about 65%. In some embodiments, the reduction in soluble liver Z-AAT after administering ADS-001 or a salt thereof to a subject in need thereof is about 65%. In some embodiments, the reduction in soluble liver Z-AAT after administering ADS-001 or a salt thereof to a subject in need thereof is at least about 70%. In some embodiments, the reduction in soluble liver Z-AAT after administering ADS-001 or a salt thereof to a subject in need thereof is about 70%. In some embodiments, the reduction in soluble liver Z-AAT after administering ADS-001 or a salt thereof to a subject in need thereof is at least about 75%. In some embodiments, the reduction in soluble liver Z-AAT after administering ADS-001 or a salt thereof to a subject in need thereof is about 75%. In some embodiments, the reduction in soluble liver Z-AAT after administering ADS-001 or a salt thereof to a subject in need thereof is at least about 80%. In some embodiments, the reduction in soluble liver Z-AAT after administering ADS-001 or a salt thereof to a subject in need thereof is about 80%. In some embodiments, the reduction in soluble liver Z-AAT after administering ADS-001 or a salt thereof to a subject in need thereof is at least about 85%. In some embodiments, the reduction in soluble liver Z-AAT after administering ADS-001 or a salt thereof to a subject in need thereof is about 85%. In some embodiments, the reduction in soluble liver Z-AAT after administering ADS-001 or a salt thereof to a subject in need thereof is at least about 90%. In some embodiments, the reduction in soluble liver Z-AAT after administering ADS-001 or a salt thereof to a subject in need thereof is about 90%. In some embodiments, the reduction in soluble liver Z-AAT after administering ADS-001 or a salt thereof to a subject in need thereof is at least about 95%. In some embodiments, the reduction in soluble liver Z-AAT after administering ADS-001 or a salt thereof to a subject in need thereof is about 95%. In some embodiments, the reduction in soluble liver Z-AAT is between about 50% and about 97%.

In some embodiments, the liver Z-AAT insoluble or polymer protein level in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater than about 99% relative to the subject prior to being administered the AAT RNAi Drug Substance or to a subject not receiving the AAT RNAi Drug Substance.

In some embodiments, the liver Z-AAT insoluble or polymer protein level in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or greater than about 99% relative to the subject prior to being administered the AAT RNAi Drug Substance or to a subject not receiving the AAT RNAi Drug Substance.

In some embodiments, the liver Z-AAT insoluble or polymer protein level in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by between about 5% and about 10%, between about 10% and about 15%, between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, or between about 95% and 100% relative to the subject prior to being administered the AAT RNAi Drug Substance or to a subject not receiving the AAT RNAi Drug Substance.

In some embodiments, the reduction in insoluble liver Z-AAT after administering ADS-001 or a salt thereof to a subject in need thereof is at least about 40%. In some embodiments, the reduction in insoluble liver Z-AAT after administering ADS-001 or a salt thereof to a subject in need thereof is about 40%. In some embodiments, the reduction in insoluble liver Z-AAT after administering ADS-001 or a salt thereof to a subject in need thereof is at least about 45%. In some embodiments, the reduction in insoluble liver Z-AAT after administering ADS-001 or a salt thereof to a subject in need thereof is about 45%. In some embodiments, the reduction in insoluble liver Z-AAT after administering ADS-001 or a salt thereof to a subject in need thereof is at least about 50%. In some embodiments, the reduction in insoluble liver Z-AAT after administering ADS-001 or a salt thereof to a subject in need thereof is about 50%. In some embodiments, the reduction in insoluble liver Z-AAT after administering ADS-001 or a salt thereof to a subject in need thereof is at least about 55%. In some embodiments, the reduction in insoluble liver Z-AAT after administering ADS-001 or a salt thereof to a subject in need thereof is about 55%. In some embodiments, the reduction in insoluble liver Z-AAT after administering ADS-001 or a salt thereof to a subject in need thereof is at least about 60%. In some embodiments, the reduction in insoluble liver Z-AAT after administering ADS-001 or a salt thereof to a subject in need thereof is about 60%. In some embodiments, the reduction in insoluble liver Z-AAT after administering ADS-001 or a salt thereof to a subject in need thereof is at least about 65%. In some embodiments, the reduction in insoluble liver Z-AAT after administering ADS-001 or a salt thereof to a subject in need thereof is about 65%. In some embodiments, the reduction in insoluble liver Z-AAT after administering ADS-001 or a salt thereof to a subject in need thereof is at least about 70%. In some embodiments, the reduction in insoluble liver Z-AAT after administering ADS-001 or a salt thereof to a subject in need thereof is about 70%. In some embodiments, the reduction in insoluble liver Z-AAT after administering ADS-001 or a salt thereof to a subject in need thereof is at least about 75%. In some embodiments, the reduction in insoluble liver Z-AAT after administering ADS-001 or a salt thereof to a subject in need thereof is about 75%. In some embodiments, the reduction in insoluble liver Z-AAT after administering ADS-001 or a salt thereof to a subject in need thereof is at least about 80%. In some embodiments, the reduction in insoluble liver Z-AAT after administering ADS-001 or a salt thereof to a subject in need thereof is about 80%. In some embodiments, the reduction in insoluble liver Z-AAT after administering ADS-001 or a salt thereof to a subject in need thereof is at least about 85%. In some embodiments, the reduction in insoluble liver Z-AAT after administering ADS-001 or a salt thereof to a subject in need thereof is about 85%. In some embodiments, the reduction in insoluble liver Z-AAT after administering ADS-001 or a salt thereof to a subject in need thereof is at least about 90%. In some embodiments, the reduction in insoluble liver Z-AAT after administering ADS-001 or a salt thereof to a subject in need thereof is about 90%. In some embodiments, the reduction in insoluble liver Z-AAT after administering ADS-001 or a salt thereof to a subject in need thereof is at least about 95%. In some embodiments, the reduction in insoluble liver Z-AAT after administering ADS-001 or a salt thereof to a subject in need thereof is about 95%. In some embodiments, the reduction in insoluble liver Z-AAT after administering ADS-001 or a salt thereof to a subject in need thereof is between about 40% and about 97%.

In some embodiments, both the liver Z-AAT insoluble or polymer protein level and the Z-AAT soluble or monomer protein level in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater than about 99% relative to the subject prior to being administered the AAT RNAi Drug Substance or to a subject not receiving the AAT RNAi Drug Substance.

In some embodiments, both the liver Z-AAT insoluble or polymer protein level and the Z-AAT soluble or monomer protein level in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or greater than about 99% relative to the subject prior to being administered the AAT RNAi Drug Substance or to a subject not receiving the AAT RNAi Drug Substance.

In some embodiments, both the liver Z-AAT insoluble or polymer protein level and the Z-AAT soluble or monomer protein level in a subject to whom an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, has been administered is reduced by between about 5% and about 10%, between about 10% and about 15%, between about 15% and about 20%, between about 20% and about 25%, between about 25% and about 30%, between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, or between about 95% and 100% relative to the subject prior to being administered the AAT RNAi Drug Substance or to a subject not receiving the AAT RNAi Drug Substance.

In some embodiments, the administration of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof to a subject in need thereof results in a reduction in serum Z-AAT of at least about 70% relative to the subject prior to being administered the AAT RNAi Drug Substance or to a subject not receiving the AAT RNAi Drug Substance.

In some embodiments, the reduction in serum Z-AAT after administering ADS-001 or a salt thereof to a subject in need thereof is at least about 70%. In some embodiments, the reduction in serum Z-AAT after administering ADS-001 or a salt thereof to a subject in need thereof is about 70%. In some embodiments, the reduction in serum Z-AAT after administering ADS-001 or a salt thereof to a subject in need thereof is at least about 75%. In some embodiments, the reduction in serum Z-AAT after administering ADS-001 or a salt thereof to a subject in need thereof is about 75%. In some embodiments, the reduction in serum Z-AAT after administering ADS-001 or a salt thereof to a subject in need thereof is at least about 80%. In some embodiments, the reduction in serum Z-AAT after administering ADS-001 or a salt thereof to a subject in need thereof is about 80%. In some embodiments, the reduction in serum Z-AAT after administering ADS-001 or a salt thereof to a subject in need thereof is at least about 85%. In some embodiments, the reduction in serum Z-AAT after administering ADS-001 or a salt thereof to a subject in need thereof is about 85%. In some embodiments, the reduction in serum Z-AAT after administering ADS-001 or a salt thereof to a subject in need thereof is at least about 90%. In some embodiments, the reduction in serum Z-AAT after administering ADS-001 or a salt thereof to a subject in need thereof is about 90%. In some embodiments, the reduction in serum Z-AAT after administering ADS-001 or a salt thereof to a subject in need thereof is at least about 95%. In some embodiments, the reduction in serum Z-AAT after administering ADS-001 or a salt thereof to a subject in need thereof is about 95%. In some embodiments, the reduction in serum Z-AAT after administering ADS-001 or a salt thereof to a subject in need thereof is about 100%. In some embodiments, the reduction in serum Z-AAT after administering ADS-001 or a salt thereof to a subject in need thereof is between about 70% and about 100%.

In some embodiments, the administration of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof to a subject in need thereof results in a reduction in total liver Z-AAT of at least about 70% relative to the subject prior to being administered the AAT RNAi Drug Substance or to a subject not receiving the AAT RNAi Drug Substance.

In some embodiments, the reduction in total liver Z-AAT after administering ADS-001 or a salt thereof to a subject in need thereof is at least about 70%. In some embodiments, the reduction in total liver Z-AAT after administering ADS-001 or a salt thereof to a subject in need thereof is about 70%. In some embodiments, the reduction in total liver Z-AAT after administering ADS-001 or a salt thereof to a subject in need thereof is at least about 75%. In some embodiments, the reduction in total liver Z-AAT after administering ADS-001 or a salt thereof to a subject in need thereof is about 75%. In some embodiments, the reduction in total liver Z-AAT after administering ADS-001 or a salt thereof to a subject in need thereof is at least about 80%. In some embodiments, the reduction in total liver Z-AAT after administering ADS-001 or a salt thereof to a subject in need thereof is about 80%. In some embodiments, the reduction in total liver Z-AAT after administering ADS-001 or a salt thereof to a subject in need thereof is at least about 85%. In some embodiments, the reduction in total liver Z-AAT after administering ADS-001 or a salt thereof to a subject in need thereof is about 85%. In some embodiments, the reduction in total liver Z-AAT after administering ADS-001 or a salt thereof to a subject in need thereof is at least about 90%. In some embodiments, the reduction in total liver Z-AAT after administering ADS-001 or a salt thereof to a subject in need thereof is about 90%. In some embodiments, the reduction in total liver Z-AAT after administering ADS-001 or a salt thereof to a subject in need thereof is at least about 95%. In some embodiments, the reduction in total liver Z-AAT after administering ADS-001 or a salt thereof to a subject in need thereof is about 95%. In some embodiments, the reduction in total liver Z-AAT after administering ADS-001 or a salt thereof to a subject in need thereof is about 100%. In some embodiments, the reduction in total liver Z-AAT after administering ADS-001 or a salt thereof to a subject in need thereof is between about 70% and about 100%.

In some embodiments, the administration of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof to a subject in need thereof results in a reduction in ALT is at least about 30% relative to the subject prior to being administered the AAT RNAi Drug Substance or to a subject not receiving the AAT RNAi Drug Substance.

In some embodiments, the reduction in ALT after administering ADS-001 or a salt thereof to a subject in need thereof is at least about 30%. In some embodiments, the reduction in ALT after administering ADS-001 or a salt thereof to a subject in need thereof is about 30%. In some embodiments, the reduction in ALT after administering ADS-001 or a salt thereof to a subject in need thereof is at least about 35%. In some embodiments, the reduction in ALT after administering ADS-001 or a salt thereof to a subject in need thereof is about 35%. In some embodiments, the reduction in ALT after administering ADS-001 or a salt thereof to a subject in need thereof is at least about 40%. In some embodiments, the reduction in ALT after administering ADS-001 or a salt thereof to a subject in need thereof is about 40%. In some embodiments, the reduction in ALT after administering ADS-001 or a salt thereof to a subject in need thereof is at least about 45%. In some embodiments, the reduction in ALT after administering ADS-001 or a salt thereof to a subject in need thereof is about 45%. In some embodiments, the reduction in ALT after administering ADS-001 or a salt thereof to a subject in need thereof is at least about 50%. In some embodiments, the reduction in ALT after administering ADS-001 or a salt thereof to a subject in need thereof is about 50%. In some embodiments, the reduction in ALT after administering ADS-001 or a salt thereof to a subject in need thereof is between about 30% and about 50%.

In some embodiments, the administration of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof to a subject in need thereof results in a reduction in GGT is at least about 25% relative to the subject prior to being administered the AAT RNAi Drug Substance or to a subject not receiving the AAT RNAi Drug Substance.

In some embodiments, the reduction in GGT after administering ADS-001 or a salt thereof to a subject in need thereof is at least about 25%. In some embodiments, the reduction in GGT after administering ADS-001 or a salt thereof to a subject in need thereof is about 25%. In some embodiments, the reduction in GGT after administering ADS-001 or a salt thereof to a subject in need thereof is at least about 30%. In some embodiments, the reduction in GGT after administering ADS-001 or a salt thereof to a subject in need thereof is about 30%. In some embodiments, the reduction in GGT after administering ADS-001 or a salt thereof to a subject in need thereof is at least about 35%. In some embodiments, the reduction in GGT after administering ADS-001 or a salt thereof to a subject in need thereof is about 35%. In some embodiments, the reduction in GGT after administering ADS-001 or a salt thereof to a subject in need thereof is at least about 40%. In some embodiments, the reduction in GGT after administering ADS-001 or a salt thereof to a subject in need thereof is about 40%. In some embodiments, the reduction in GGT after administering ADS-001 or a salt thereof to a subject in need thereof is at least about 45%. In some embodiments, the reduction in GGT after administering ADS-001 or a salt thereof to a subject in need thereof is about 45%. In some embodiments, the reduction in GGT after administering ADS-001 or a salt thereof to a subject in need thereof is between about 25% and about 45%.

In some embodiments, the administration of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof to a subject in need thereof results in a reduction in Pro-C3 is at least about 15% relative to the subject prior to being administered the AAT RNAi Drug Substance or to a subject not receiving the AAT RNAi Drug Substance.

In some embodiments, the reduction in Pro-C3 after administering ADS-001 or a salt thereof to a subject in need thereof is at least about 15%. In some embodiments, the reduction in Pro-C3 after administering ADS-001 or a salt thereof to a subject in need thereof is about 15%. In some embodiments, the reduction in Pro-C3 after administering ADS-001 or a salt thereof to a subject in need thereof is at least about 20%. In some embodiments, the reduction in Pro-C3 after administering ADS-001 or a salt thereof to a subject in need thereof is about 20%. In some embodiments, the reduction in Pro-C3 after administering ADS-001 or a salt thereof to a subject in need thereof is at least about 25%. In some embodiments, the reduction in Pro-C3 after administering ADS-001 or a salt thereof to a subject in need thereof is about 25%. In some embodiments, the reduction in Pro-C3 after administering ADS-001 or a salt thereof to a subject in need thereof is at least about 30%. In some embodiments, the reduction in Pro-C3 after administering ADS-001 or a salt thereof to a subject in need thereof is about 30%. In some embodiments, the reduction in Pro-C3 after administering ADS-001 or a salt thereof to a subject in need thereof is at least about 35%. In some embodiments, the reduction in Pro-C3 after administering ADS-001 or a salt thereof to a subject in need thereof is about 35%. In some embodiments, the reduction in Pro-C3 after administering ADS-001 or a salt thereof to a subject in need thereof is at least about 40%. In some embodiments, the reduction in Pro-C3 after administering ADS-001 or a salt thereof to a subject in need thereof is about 40%. In some embodiments, the reduction in Pro-C3 after administering ADS-001 or a salt thereof to a subject in need thereof is at least about 45%. In some embodiments, the reduction in Pro-C3 after administering ADS-001 or a salt thereof to a subject in need thereof is about 45%. In some embodiments, the reduction in Pro-C3 after administering ADS-001 or a salt thereof to a subject in need thereof is at least about 50%. In some embodiments, the reduction in Pro-C3 after administering ADS-001 or a salt thereof to a subject in need thereof is about 50%. In some embodiments, the reduction in Pro-C3 after administering ADS-001 or a salt thereof to a subject in need thereof is at least about 55%. In some embodiments, the reduction in Pro-C3 after administering ADS-001 or a salt thereof to a subject in need thereof is about 55%. In some embodiments, the reduction in Pro-C3 after administering ADS-001 or a salt thereof to a subject in need thereof is at least about 60%. In some embodiments, the reduction in Pro-C3 after administering ADS-001 or a salt thereof to a subject in need thereof is about 60%. In some embodiments, the reduction in Pro-C3 after administering ADS-001 or a salt thereof to a subject in need thereof is at least about 65%. In some embodiments, the reduction in Pro-C3 after administering ADS-001 or a salt thereof to a subject in need thereof is about 65%. In some embodiments, the reduction in Pro-C3 after administering ADS-001 or a salt thereof to a subject in need thereof is at least about 70%. In some embodiments, the reduction in Pro-C3 after administering ADS-001 or a salt thereof to a subject in need thereof is about 70%. In some embodiments, the reduction in Pro-C3 after administering ADS-001 or a salt thereof to a subject in need thereof is at least about 75%. In some embodiments, the reduction in Pro-C3 after administering ADS-001 or a salt thereof to a subject in need thereof is about 75%. In some embodiments, the reduction in Pro-C3 after administering ADS-001 or a salt thereof to a subject in need thereof is at least about 80%. In some embodiments, the reduction in Pro-C3 after administering ADS-001 or a salt thereof to a subject in need thereof is about 80%. In some embodiments, the reduction in Pro-C3 after administering ADS-001 or a salt thereof to a subject in need thereof is at least about 85%. In some embodiments, the reduction in Pro-C3 after administering ADS-001 or a salt thereof to a subject in need thereof is about 85%. In some embodiments, the reduction in Pro-C3 after administering ADS-001 or a salt thereof to a subject in need thereof is at least about 90%. In some embodiments, the reduction in Pro-C3 after administering ADS-001 or a salt thereof to a subject in need thereof is about 90%. In some embodiments, the reduction in Pro-C3 after administering ADS-001 or a salt thereof to a subject in need thereof is between about 15% and about 90%.

In some embodiments, the administration of an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof to a subject in need thereof results in a reduction in fibrosis is at least about 15% as measured by FIBROSCAN® relative to the subject prior to being administered the AAT RNAi Drug Substance or to a subject not receiving the AAT RNAi Drug Substance.

In some embodiments, the reduction in fibrosis after administering ADS-001 or a salt thereof to a subject in need thereof is at least about 15% as measured by FIBROSCAN®. In some embodiments, the reduction in fibrosis after administering ADS-001 or a salt thereof to a subject in need thereof is about 15% as measured by FIBROSCAN®. In some embodiments, the reduction in fibrosis after administering ADS-001 or a salt thereof to a subject in need thereof is at least about 20% as measured by FIBROSCAN®. In some embodiments, the reduction in fibrosis after administering ADS-001 or a salt thereof to a subject in need thereof is about 20% as measured by FIBROSCAN®. In some embodiments, the reduction in fibrosis after administering ADS-001 or a salt thereof to a subject in need thereof is at least about 25% as measured by FIBROSCAN®. In some embodiments, the reduction in fibrosis after administering ADS-001 or a salt thereof to a subject in need thereof is about 25% as measured by FIBROSCAN®. In some embodiments, the reduction in fibrosis after administering ADS-001 or a salt thereof to a subject in need thereof is at least about 30% as measured by FIBROSCAN®. In some embodiments, the reduction in fibrosis after administering ADS-001 or a salt thereof to a subject in need thereof is about 30% as measured by FIBROSCAN®. In some embodiments, the reduction in fibrosis after administering ADS-001 or a salt thereof to a subject in need thereof is at least about 35% as measured by FIBROSCAN®. In some embodiments, the reduction in fibrosis after administering ADS-001 or a salt thereof to a subject in need thereof is about 35% as measured by FIBROSCAN®. In some embodiments, the reduction in fibrosis after administering ADS-001 or a salt thereof to a subject in need thereof is at least about 40% as measured by FIBROSCAN®. In some embodiments, the reduction in fibrosis after administering ADS-001 or a salt thereof to a subject in need thereof is about 40% as measured by FIBROSCAN®. In some embodiments, the reduction in fibrosis after administering ADS-001 or a salt thereof to a subject in need thereof is at least about 45% as measured by FIBROSCAN(R). In some embodiments, the reduction in fibrosis after administering ADS-001 or a salt thereof to a subject in need thereof is about 45% as measured by FIBROSCAN®. In some embodiments, the reduction in fibrosis after administering ADS-001 or a salt thereof to a subject in need thereof is at least about 50% as measured by FIBROSCAN®. In some embodiments, the reduction in fibrosis after administering ADS-001 or a salt thereof to a subject in need thereof is about 50% as measured by FIBROSCAN®. In some embodiments, the reduction in fibrosis after administering ADS-001 or a salt thereof to a subject in need thereof is at least about 55% as measured by FIBROSCAN®. In some embodiments, the reduction in fibrosis after administering ADS-001 or a salt thereof to a subject in need thereof is about 55% as measured by FIBROSCAN®. In some embodiments, the reduction in fibrosis after administering ADS-001 or a salt thereof to a subject in need thereof is at least about 60% as measured by FIBROSCAN®. In some embodiments, the reduction in fibrosis after administering ADS-001 or a salt thereof to a subject in need thereof is about 60% as measured by FIBROSCAN®. In some embodiments, the reduction in fibrosis after administering ADS-001 or a salt thereof to a subject in need thereof is at least about 65% as measured by FIBROSCAN®. In some embodiments, the reduction in fibrosis after administering ADS-001 or a salt thereof to a subject in need thereof is about 65% as measured by FIBROSCAN®. In some embodiments, the reduction in fibrosis after administering ADS-001 or a salt thereof to a subject in need thereof is at least about 70% as measured by FIBROSCAN®. In some embodiments, the reduction in fibrosis after administering ADS-001 or a salt thereof to a subject in need thereof is about 70% as measured by FIBROSCAN®. In some embodiments, the reduction in fibrosis after administering ADS-001 or a salt thereof to a subject in need thereof is at least about 75% as measured by FIBROSCAN®. In some embodiments, the reduction in fibrosis after administering ADS-001 or a salt thereof to a subject in need thereof is about 75% as measured by FIBROSCAN®. In some embodiments, the reduction in fibrosis after administering ADS-001 or a salt thereof to a subject in need thereof is at least about 80% as measured by FIBROSCAN®. In some embodiments, the reduction in fibrosis after administering ADS-001 or a salt thereof to a subject in need thereof is about 80% as measured by FIBROSCAN®. In some embodiments, the reduction in fibrosis after administering ADS-001 or a salt thereof to a subject in need thereof is at least about 85% as measured by FIBROSCAN®. In some embodiments, the reduction in fibrosis after administering ADS-001 or a salt thereof to a subject in need thereof is about 85% as measured by FIBROSCAN®. In some embodiments, the reduction in fibrosis after administering ADS-001 or a salt thereof to a subject in need thereof is at least about 90% as measured by FIBROSCAN®. In some embodiments, the reduction in fibrosis after administering ADS-001 or a salt thereof to a subject in need thereof is about 90% as measured by FIBROSCAN®. In some embodiments, the reduction in fibrosis after administering ADS-001 or a salt thereof to a subject in need thereof is between about 15% and about 90% as measured by FIBROSCAN®.

A reduction in AAT gene expression (including PiZZ genotype), AAT mRNA (including PiZZ genotype), or AAT protein levels (including Z-AAT protein) can be assessed and quantified by general methods known in the art. The Examples disclosed herein forth generally known methods for assessing inhibition of AAT gene expression and reduction in AAT protein levels. The reduction or decrease in AAT mRNA level and/or protein level (including Z-AAT soluble and insoluble protein levels) are collectively referred to herein as a reduction or decrease in AAT or inhibiting or reducing the expression of AAT.

All the changes described above, e.g., changes in periportal hepatocytes; serum Z-AAT; liver Z-AAT; soluble liver Z-AAT; insoluble liver Z-AAT; ALT; GGT; Pro-C3; or steatosis, are with respect to a predetermined threshold, to the levels in the subject prior to being administered the AAT RNAi Drug Substance, to the levels in a subject not receiving the AAT RNAi Drug Substance, or to control levels determined in a population. All the measurements described above for, e.g., fibrosis; periportal hepatocytes; serum Z-AAT; liver Z-AAT; soluble liver Z-AAT; insoluble liver Z-AAT; ALT; GGT; Pro-C3; or steatosis, are conducted as described in the present disclosure or using methods known in the art.

As used herein, the terms “amount of liver Z-AAT protein,” “liver Z-AAT protein level,” “liver Z-AAT protein burden,” refer to the quantity of Z-AAT protein that is measured in the liver of a human subject, and unless expressly indicated otherwise such terms are used interchangeably herein. As disclosed more fully in the non-limiting Examples herein, liver biopsies can be taken of subjects and those samples can be homogenized and then assessed for the total amount of Z-AAT protein present. The amount of soluble Z-AAT protein present (predominantly monomer form Z-AAT protein) can similarly be quantified, and the level of insoluble (polymer) Z-AAT protein present can then be calculated by subtracting the soluble quantity from the total.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition of the number, severity, and/or frequency of one or more symptoms of a disease in a subject.

As used herein, “monthly dosing” or “monthly” administration means every 28 days. As used herein, “quarterly dosing” or “quarterly” administration means every 84 days. The term “about” when used in connection with monthly dosing means monthly dosing +/- 3 days. The term “about” when used in connection with quarterly dosing means quarterly dosing +/- 9 days. The term “about” when used in connection with a number of weeks of dosing means +/- 1 week.

As used herein, the phrase “introducing into a cell,” when referring to an AAT RNAi Drug Substance disclosed herein, e.g., a dsRNA comprising a sense strand consisting of SEQ ID NO: 6 and an antisense strand consisting of SEQ ID NO: 2 such as ADS-001 or a salt thereof, means functionally delivering the RNAi agent into a cell. The phrase “functional delivery,” means that delivering the RNAi agent to the cell in a manner that enables the RNAi agent to have the expected biological activity, e.g., sequence-specific inhibition of gene expression.

Unless stated otherwise, use of the symbol

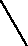

as used herein means that any group or groups may be linked thereto that is in accordance with the scope of the inventions described herein.

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

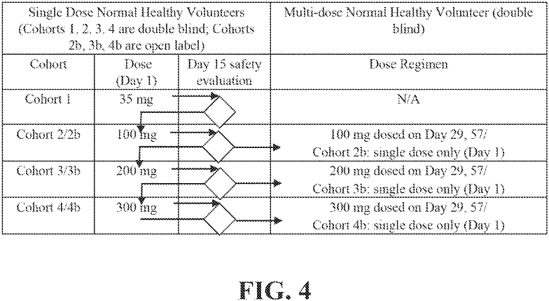

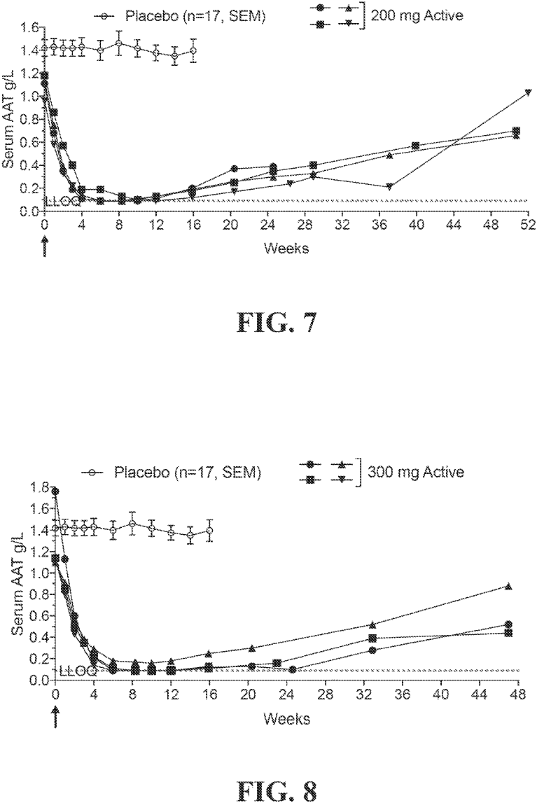

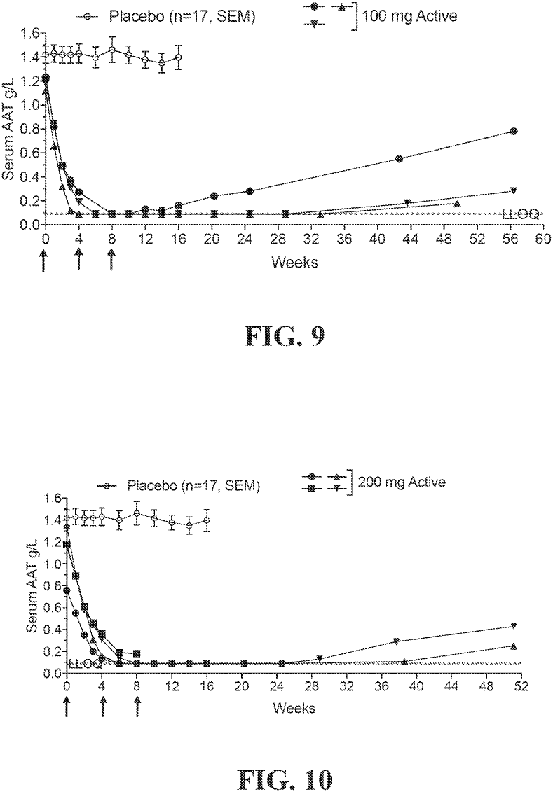

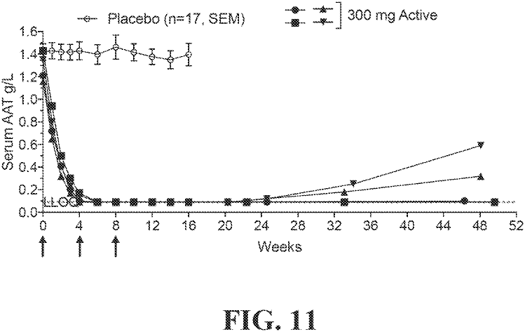

In International Patent Application No. PCT/US20/36359, entitled Methods For The Treatment Of Alpha-1 Antitrypsin Deficiency (AATD), Applicants described data from a Phase 1 Study of AAT RNAi Drug Substance (ADS-001) in normal healthy volunteers (HBVs). For completeness, that information is set forth in Example 2 and FIGS. 4-11 hereto. In the present application, Applicants set forth initial data from an open-label Phase 2 study in human subjects diagnosed with AATD. Surprisingly and unexpectedly, after just 6 months and three doses of AAT RNAi Drug Substance (ADS-001), a reduction in liver Z-AAT protein levels were evident.

The above provided embodiments and items are now illustrated with the following, non-limiting examples.

### EXAMPLES

### Example 1. Synthesis and Formulation of AAT RNAi Drug Substance (ADS-001)

The AAT RNAi Drug Substance suitable for use in the methods disclosed herein can be synthesized using standard phosphoramidite technology on solid phase oligonucleotide synthesis as is known in the art. Commercially available oligonucleotide synthesizers (e.g., MERMADE96E® (Bioautomation) or MERMADE12® (Bioautomation)) may be used. Syntheses can be performed on a solid support made of controlled pore glass (CPG, 500 Å or 600 Å, obtained from Prime Synthesis, Aston, PA, USA). The monomer positioned at the 3′ end of the respective strand may be attached to the solid support as a starting point for synthesis. All RNA, 2′-modified RNA phosphoramidites, and inverted abasic phosphoramidites can be purchased commercially. Targeting group-containing phosphoramidites can be synthesized that are suitable for addition to the 5′ end of the sense strand. Standard cleavage, deprotection, purification, and annealing steps can be utilized as is known in the art. Further description related to the synthesis of AAT RNAi agents may be found, for example, in International Patent Application Publication No. WO 2018/132432 (Application No. PCT/US2018/013102) and WO 2018/044350 (PCT/US2017/021147), each of which is incorporated by reference herein in its entirety. AAT RNAi Drug Substance can then be formulated by dissolving in standard pharmaceutically acceptable excipients that are generally known in the art. For example, Table 3.1 and Table 3.2 show a Formulated AAT RNAi Drug Substance that is suitable for use in the methods disclosed herein.

### Example 2. Phase I Clinical Trial of AAT RNAi Drug Substance (ADS-001) In Normal Healthy Human Volunteers (NHV).

The following example was previously presented in International Patent Application No. PCT/US20/36359, entitled Methods For The Treatment Of Alpha-1 Antitrypsin Deficiency (AATD), and Applicant wishes to reiterate that information here for completeness.

A Phase 1, single and multiple dose-escalating dose study to evaluate the safety, tolerability, pharmacokinetics and effect of AAT RNAi Drug Substance (ADS-001) on serum AAT levels in healthy volunteers (NHV) was conducted. The study subject population included healthy adult males and females 18-52 years old with a BMI between 19.0 and 35.0 kg/m².

NHV subjects were divided into a total of seven cohorts. Cohorts 1 through 4 were randomized to receive AAT RNAi Drug Substance or placebo (4 active: 4 placebo) at single escalating doses of 35 mg (Cohort 1) and multiple escalating doses of 100 mg (Cohort 2), 200 mg (Cohort 3) and 300 mg (Cohort 4) administered as a subcutaneous injection. Cohorts 1 through 4 were double-blinded. Cohorts 2b, 3b and 4b were open label consisting of 4 subjects receiving single-doses of 100, 200, and 300 mg of AAT RNAi Drug Substance. A total of 44 subjects completed the study. FIG. 4 shows the final study design for the Phase I Clinical Trial. The study parameters are summarized in the following Table 4.

TABLE 4

Phase I Clinical Study Parameters

| | |
|---|---|
| Development Phase | Phase 1: First-in-Human |
| Study Objectives | Primary Objectives: |
| | To determine the incidence and frequency of adverse events possibly or probably related to treatment as a measure of the safety and tolerability of AAT RNAi Drug Substance (ADS-001) using |
| | escalating single doses and escalating multiple doses in normal healthy human volunteers (NHV). |
| | Secondary Objectives: |
| | To evaluate the single-dose and multi-dose pharmacokinetics of AAT RNAi Drug Substance in NHV. |
| | To determine the reduction in serum AAT in response to AAT RNAi Drug Substance as a measure of drug activity. |
| | Exploratory Objectives: |
| | To evaluate the effect of single doses of AAT RNAi Drug Substance on cytokines (Cytokine panel A: interleukin-6 [IL-6], monocyte chemoattractant protein-1 [MCP-1], tumor necrosis factor-alpha [TNF-alpha], interleukin-8 [IL-8], interleukin-1beta [IL-1beta], interferon alpha [IFN alpha], IL-10, IL-12 [p40], IL-12 [p70], macrophage inflammatory protein-1alpha [Mip-1alpha]) in NHV. |
| | To evaluate the effect of single escalating doses of AAT RNAi Drug Substance on complement factors Bb, CH50, C5a, C4a, and C3a in NHV. |
| | To collect plasma samples in NHV for subsequent metabolite identification (reported in a separate report outside of this study). |
| | To collect urine samples in NHV for subsequent determination of urinary excretion and metabolite identification (reported in a separate report outside of this study). |
| Study Design | Cohorts 1 through 4: randomized, double-blind, placebo-controlled Cohorts 2b, 3b, and 4b: open label |
| Study Population | This study was conducted in NHVs, adult males and females, aged 18-52 years with BMI between 19.0 and 35.0 kg/m². |
| Investigational Product | AAT RNAi Drug Substance (ADS-001) (see Table 2), administered as Formulated AAT RNAi Drug Substance (ADS-001-1) (see Table 3.1) |
| Dosage and Frequency | Cohort 1: randomized to receive AAT RNAi Drug Substance (ADS-001) or placebo (4 active: 4 placebo) at a single dose of 35 mg administered as a single subcutaneous injection. |
| | Cohorts 2-4: randomized to receive three monthly (i.e., days 1, 29, and 57) doses of 100 mg (Cohort 2), 200 mg (Cohort 3), or 300 mg (Cohort 4) AAT RNAi Drug Substance or placebo (4 active: 4 placebo), via subcutaneous injection. |
| | Cohorts 2b, 3b, and 4b: enrolled to receive a single dose of 100 mg (Cohort 2b), 200 mg (Cohort 3b), or 300 mg (Cohort 4b) AAT RNAi |
| | Drug Substance (4 active) administered as a single subcutaneous injection. |
| | As noted immediately above, all subcutaneous injections of AAT RNAi Drug Substance were administered as Formulated AAT RNAi Drug Substance (ADS-001-1) (see Table 3.1) |
| Reference Formulation | Placebo (PBO): normal saline (0.9%) administered subcutaneously with matching volume. |
| Safety Evaluation Criteria | Safety was assessed by adverse events, serious adverse events, physical examinations, vital sign measurements (blood pressure, heart rate, temperature, and respiratory rate), resting ECG measurements, clinical laboratory tests, concomitant medications/therapy, injection site reactions (ISRs), reasons for treatment discontinuation, and 90-day post-Day 29 (Cohort 1) and post-Day 113 (all other cohorts) pregnancy follow up. |
| Pharmacokinetics Evaluation | Blood samples will be collected from each subject for pharmacokinetic analysis after dose 1 (Cohort 1) and after dose 1 and 3 (Cohorts 2, 3, and 4) |
| Data Analysis | Screening, Compliance, Tolerability and Safety Data: |
| | Safety analyses will be performed, and the results summarized by cohort. The incidence and frequency of adverse events (AEs), serious adverse events (SAEs), related AEs, related SAEs, and AEs leading to discontinuation, will be summarized by cohort per SOC, PT, and severity. Other safety parameters will be summarized at each scheduled time. |
| | Pharmacokinetics (NHV subjects only): |
| | Plasma concentrations of AAT RNAi Drug Substance constituents will be used to calculate the following PK parameters: maximum observed plasma concentration (Cmax), area under the plasma concentration time curve (AUC) from time 0 to 24 hours (AUC0-24), AUC from time 0 extrapolated to infinity (AUCinf), and terminal elimination half-life (t½). Pharmacokinetic parameters will be determined using non-compartmental methods. Descriptive statistics of PK parameters will include mean, standard deviation (SD), coefficient of variation, median, minimum, and maximum. PK results will be analyzed for dose proportionality, and sex differences. |

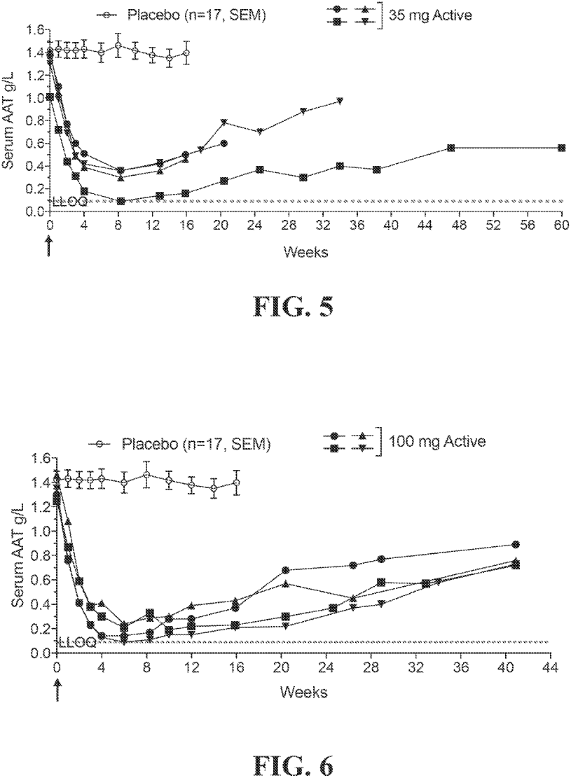

Serum AAT reduction results from the study showed that administration of AAT RNAi Drug Substance at doses from 35 to 300 mg resulted in deep reduction of serum AAT when compared with placebo. Initially, a cohort was proposed as part of the clinical trial protocol at 400 mg of AAT RNAi Drug Substance per dose. However, in view of the unexpected potency at the 35, 100, 200, and 300 mg doses, the 400 mg cohort was removed from the study protocol. Doses of 35 mg, 100 mg, and 200 mg yielded substantial serum AAT reductions, with both 100 mg and 200 mg reaching approximately 90% mean serum AAT reduction after multiple doses in the Phase I study. FIGS. 5 through 11 report on the serum AAT reductions of the various cohorts in the Phase I study.

There was no clear dose-dependent response across all dose levels because, surprisingly and unexpectedly, the dose levels at 100 mg and 200 mg produced substantial (reaching approximately 90%) and similar knockdown to the higher 300 mg dose. While the lowest dose of 35 mg was still quite active, it was not as active as 100 mg administered as a single dose, indicating a degree of dose response.

Duration of serum AAT reduction (>58%) from a single-dose of 35 mg lasted longer than initially anticipated, out to 16-weeks post dose administration with subsequent return towards baseline. For example, thirty-four weeks after the 35 mg single dose, one subject’s serum AAT level has returned to above 90 mg/dL, while a second subject’s serum AAT level remained at 40 mg/dL (60.4% reduced from baseline). There was no significant difference in the duration of response from single-doses of 100 mg to 300 mg of AAT RNAi Drug Substance, with return to baseline beginning between 8 and 16 weeks after the single-dose.

Multiple-doses of AAT RNAi Drug Substance maintain deep reduction in serum AAT for a longer duration than a single dose in general. These data suggest that a second dose received on Day 29 (i.e., after one month from an initial dose), may further reduce serum AAT levels or maintain reductions, and subsequent doses may be administered to maintain maximum reduced serum AAT every 12 weeks (i.e., quarterly).

In the Phase I study there were no deaths, no serious adverse events (SAEs), and no adverse events (AEs) rated as severe in intensity. Two subjects reported three AEs as moderate in intensity across subjects receiving AAT RNAi Drug Substance (upper respiratory tract infection, rhinorrhea, chest pain general). Three subjects reported three AEs as moderate in intensity across subjects receiving placebo (2-gastroenteritis, musculoskeletal chest pain-left sided). All other AEs have been reported as mild. The majority of subjects reported AEs not related to study treatment. One AE occurred in a subject receiving AAT which led to the premature discontinuation of therapy, although the subject continued to be followed on study. Ninety-four AEs were reported in 28 subjects receiving at least a single dose of Formulated AAT RNAi Drug Substance. Forty-six AEs were reported in 17 subjects receiving placebo. There is no clear pattern of an increased frequency or intensity of AEs with dose escalation.

Six AEs at the injection site occurred in 6 subjects across all Formulated AAT RNAi Drug Substance cohorts which all occurred in subjects receiving drug. There were no injection site AEs in placebo subjects. The injection site reactions reported included injection site bruising, erythema, and pain. These combined AEs at the injection site were reported by 21.4% of subjects receiving Formulated AAT RNAi Drug Substance. Six of 50 injections of Formulated AAT RNAi Drug Substance resulted in an injection site AE or 12%. No injection site AEs were reported more than once in a single subject. All injection site AEs have been considered mild in intensity.

### Example 3. Phase II Clinical Trial of AAT RNAi Drug Substance (ADS-001) in Patients with AAT-Associated Liver Disease.

A Pilot Open Label, Multi-dose, Phase 2 Study to Assess the Safety and Efficacy of ARO-AAT in Patients with Alpha-1 Antitrypsin Deficiency Associated Liver Disease (AATD) was conducted. The study subject population included PiZZ patients (based on genotype completed at baseline or from a source verifiable document) AAT subjects, that are 18-75 years old.

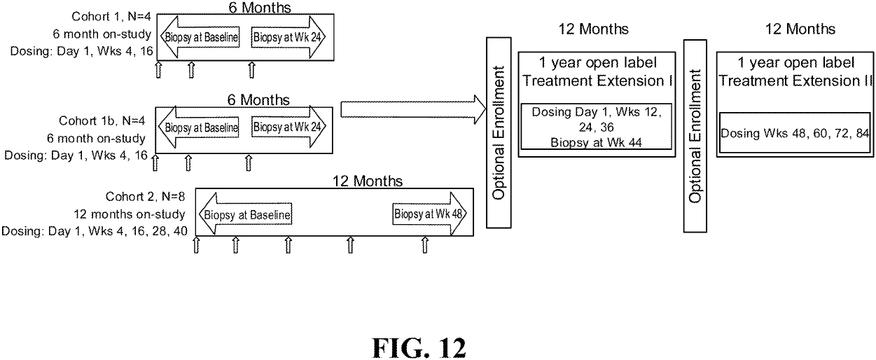

PiZZ subjects were divided into a total of three cohorts. All subjects were required to undertake a liver biopsy prior to initial dose. FIG. 12 shows the study design for the Phase II Clinical Trial.

Cohorts 1 and 1b consist of up to 4 subjects receiving a total of three doses of AAT RNAi Drug Substance at either 200 mg (Cohort 1) or 100 mg (Cohort 1b) administered as a subcutaneous injection. Doses are administered on Day 1, four weeks after the initial dose, and twelve weeks after the second dose. Twenty-four (24) weeks after the third dose (about 40 weeks or about 6 months after the initial dose), a second biopsy is taken.

Cohort 2 will consist of up to 8 subjects receiving a total of five doses of AAT RNAi Drug Substance at 200 mg administered as a subcutaneous injection. Doses are administered on Day 1, four weeks after the initial dose, and twelve weeks after the second dose, twelve weeks after the third dose, and twelve weeks after the fourth dose (i.e., Day 1, and weeks 4, 16, 28, and 40). Forty-eight (48) weeks after the third dose (about 88 weeks or about 1 year after the initial dose), a second biopsy is taken.

The study parameters are summarized in the following Table 5.

TABLE 5

Phase II Clinical Study Parameters

| | |
|---|---|
| Development Phase | Phase 2 |
| Study Objectives | Primary Objectives: |
| | To evaluate change from baseline over time in total, soluble, and insoluble Z-AAT concentrations in the liver of patients with AAT-associated liver disease |
| | Secondary Objectives: |
| | To determine the effect of multiple doses of ARO-AAT on circulating levels of Z-AAT alpha-1 antitrypsin over time versus baseline |
| | To evaluate the effect of ARO-AAT on changes in ALT over time. |
| | To evaluate the effect of ARO-AAT on changes in GGT over time. |
| | To evaluate the effect of ARO-AAT on changes in FIB4 and APRI over time. |
| | To evaluate the effect of ARO-AAT on changes in PRO-C3 over time. |
| | To evaluate the effect of ARO-AAT on changes in hepatic stiffness based on FIBROSCAN® over time versus baseline (when available). |
| | To evaluate effect of ARO-AAT on histological metrics of liver disease in patients with AAT-associated liver disease over time. |
| | To evaluate change from baseline in Metavir fibrosis score over time in ARO-AAT treated patients. |
| | To determine the incidence and frequency of adverse events possibly or probably related to treatment as a measure of the safety and tolerability of ARO-AAT. |
| | Exploratory Objectives: |
| | To evaluate the effect of ARO-AAT on changes in hepatic stiffness based on Magnetic Resonance Elastography (MRE) over time versus baseline (optional). |
| | To evaluate changes in hepatic SERPINA1 mRNA expression over time versus baseline in response to multiple doses of ARO-AAT (if sufficient sample available). |
| | To evaluate changes in liver fibrosis gene expression over time versus baseline (if scientifically feasible and sufficient sample available). |
| | To evaluate change in liver PAS+D stained globule size and number based on IHC over time versus baseline. |
| | To evaluate the effect of ARO-AAT on changes in liver collagen and iron content using biomarkers, special stains and imaging [Masson’s Trichrome, Sirius Red, Iron] (if scientifically feasible and sufficient sample available) over time versus baseline. |
| | To determine the effect of multiple doses of ARO-AAT on circulating levels of total alpha-1 antitrypsin at multiple post-dose time points versus baseline (patients on and not on AAT augmentation therapy will be evaluated separately). |
| Study Design | Open label |
| Study Population | PiZZ patients with Alpha-1 Antitrypsin Deficiency Both males and females are eligible, ages 18-75 |
| Investigational Product (ARO-AAT) | AAT RNAi Drug Substance (ADS-001) (see Table 2), administered as Formulated AAT RNAi Drug Substance (see Table 3.1) |
| Dosage and Frequency | Cohort 1: receive AAT RNAi Drug Substance (ADS-001) at a dose of 200 mg administered on Day 1, weeks 4 and 16, via subcutaneous injection. |
| | Cohort 1b: receive AAT RNAi Drug Substance (ADS-001) at a dose of 100 mg administered on Day 1, weeks 4 and 16, via subcutaneous injection. |
| | Cohort 2: receive AAT RNAi Drug Substance (ADS-001) at a dose of 200 mg administered on Day 1, weeks 4, 16, 28 and 40, via subcutaneous injection. |
| | As noted immediately above, all subcutaneous injections of AAT RNAi Drug Substance were administered as Formulated AAT RNAi Drug Substance (ADS-001-1) (see Table 3.1) |
| Safety Evaluation Criteria | Safety will be assessed by adverse events, serious adverse events, physical examinations, vital sign measurements (blood pressure, heart rate, temperature, and respiratory rate), resting ECG measurements, pulmonary function testing (Spirometry including FEV1 and DLCO), clinical laboratory tests, concomitant medications/therapy, injection site reactions (ISRs), reasons for treatment discontinuation, and 12-week post-last dose pregnancy follow up. |
| Data Analysis | Screening, Tolerability and Safety Data: |
| | Safety analyses will be performed, and the results summarized. Baseline safety assessments will be compared with measurements recorded post-baseline. The incidence and frequency of adverse events (AEs), serious adverse events (SAEs), related AEs, related SAEs, and AEs leading to withdrawal, dose modification, or treatment discontinuation, will be summarized by dose according to SOC and Preferred Terms. Other safety parameters will be summarized at each scheduled time. |
| | Pharmacodynamic Data: Descriptive summaries will be presented for primary, secondary and exploratory endpoints using appropriate methods. Histological changes including Ishak fibrosis score will be summarized at EOS as compared to Baseline. |

### Cohort 1

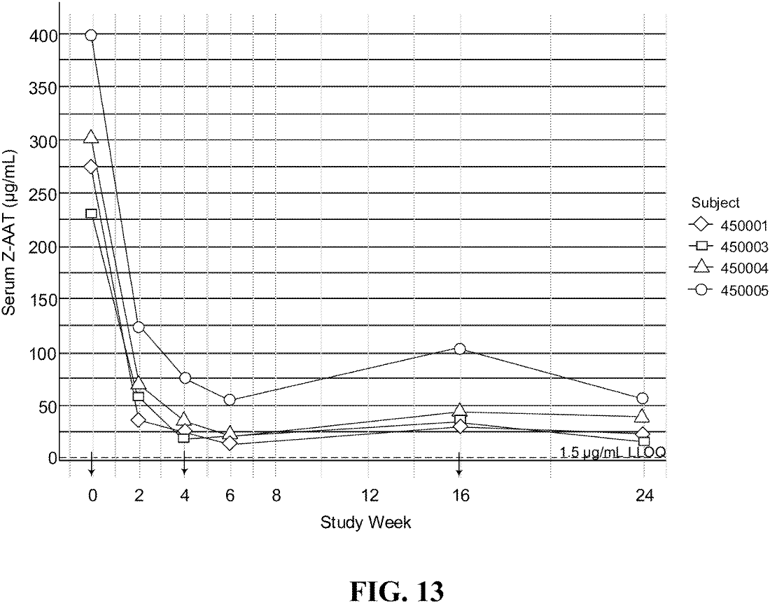

For Cohort 1, Serum Z-AAT reduction results showed that administration of AAT RNAi Drug Substance at a 200 mg dose resulted in a deep reduction of both serum and total liver Z-AAT protein levels. (See, e.g., FIG. 13).

Serum Z-AAT was measured quantitatively through a tryptic peptide that includes the Z-AAT mutation using UHPLC-MS/MS. Total and soluble liver Z-AAT protein was also measured quantitatively through a tryptic peptide that includes the Z-AAT mutation using UHPLC-MS/MS. Following homogenization of the liver tissue, the total Z-AAT protein level was determined using UHPLC-MS/MS. Centrifugation was then performed on a separate aliquot to separate the soluble and insoluble fractions. After separation, the soluble fraction was assessed for Z-AAT protein levels using UHPLC-MS/MS. The unmeasured fraction (insoluble) was derived by subtracting the soluble fraction measured from the total amount of liver Z-AAT protein determined.

Alternatively, Z-AAT protein can be quantitatively or semi-quantitatively determined by using probes or antibodies that are specific to the Z-AAT protein, using methods such as Western blot or semi-quantitative immunohistochemistry. Such methods are generally known and reagents and tools are commercially available or are otherwise within the knowledge of the person of ordinary skill in the art.

The following Table 6 shows, among other things, total liver (i.e., intra-hepatic) Z-AAT protein levels, monomer (soluble) liver Z-AAT protein levels, polymer (insoluble) liver Z-AAT protein levels, ALT enzyme levels, and GGT enzyme levels. FIBROSCAN® assessments were taken for each of the subjects both pre-dose liver biopsy and at week 24, and additional assessments were taken for each of the subjects at week 52, the results of which are shown in Table 6 as well.

TABLE 6

Relative Change From Baseline to Week 24 or Week 52 In Subjects From Cohort 1.
Subject Number

| | | | | |
|---|---|---|---|---|
| Pharmacodynamic Response | 1 | 2 | 3 | 4 |
| Δ % Serum Z-AAT | | | | |
| Week 24 | -92.3% | -93.1% | -86.7% | -85.8% |
| Week 52 | -91.0% | -94.3% | -85.2% | -77.6% |
| Total liver Z-AAT baseline^{∗}, | 33.9 | 15.2 | 35.2 | 146 |
| Δ nmol/total protein Week 24, | -26.6 | -14.4 | -25.4 | -107.2 |
| Δ % Week 24 | -78.6% | -95.1% | -72.2% | -73.4% |
| Soluble liver Z-AAT baseline^{∗}, | 16.8 | 13.6 | 33.3 | 33.5 |
| Δ nmol/total protein Week 24, | -15.1 | -12.9 | -28.9 | -27.2 |
| Δ % Week 24 | -89.8% | -94.9% | -86.8% | -81.2% |
| Δ Insoluble liver Z-AAT^{∗} Week 24, | -11.6 | -1.5 | 3.5 | -80 |
| Δ % Week 24 | -67.6% | -96.6% | 183.7% | -71.1% |
| Δ ALT | | | | |
| Week 24 | -66.4% | -54.9% | -35.7% | -50.0% |
| Week 52 | -72.7% | -52.7% | -39.3% | -59.2% |
| Δ GGT | | | | |
| Week 24 | -42.6% | -43.2% | -57.7% | -55.3% |
| Week 52 | -51.5% | -29.7% | -52.6% | -81.2% |
| Δ % FIBROSCAN® Week 24 | -25.8% | -22.4% | -0.8% | -20.9% |
| Δ Pro-C3, ng/mL Week 24 | -19.7 | 0.9 | -6.9 | -8.4 |
| Δ % Week 24 | -51.4% | 5.5% | -30.9% | -35.6% |
| Δ Pro-C3, ng/mL Week 52 | -23.4 | -4.2 | -7.0 | -9.4 |
| Δ % Week 52 | -61.1% | -25.6% | -31.4% | -39.8% |

^{∗}nmol/total protein

As shown in Table 6 above, each of the subjects attained a greater than 70% reduction in total liver Z-AAT protein levels (Δ %) and a greater than 80% reduction in monomer (soluble) liver Z-AAT protein levels. Moreover, all but one of the subjects showed a reduction in polymer (insoluble) liver Z-AAT protein levels, with 3 of the 4 subjects demonstrating reductions in Z-AAT polymer (insoluble) protein level in the range of 68-97%.

The reductions in liver Z-AAT protein levels also yielded improvements in clinically relevant biomarkers including that all subjects showed reductions in ALT ranging from 36-66%, and all subjects showed GGT reductions ranging from 43-58% compared to baseline at week 24.

All subjects further showed an improvement in FIBROSCAN® scoring with 3 of the 4 subjects demonstrating greater than 20% reductions compared to baseline at week 24. Moreover, 3 of 4 patients demonstrated reductions in the fibrogenesis biomarker Pro-C3 ranging from 31-51% at Week 24.

Histological assessments of the liver biopsies at baseline and week 24 were performed by two pathologists, with a third pathologist serving as an adjudicator in the event of any discrepancies in evaluation. At week 24, all subjects showed an improvement in at least one histological parameter when compared to their baseline biopsy, such as portal inflammation, lobular inflammation, interface hepatitis, hepatocyte cell death, and fibrosis (Metavir). Two subjects had improvement in fibrosis and two subjects had no worsening of fibrosis. All subjects showed no worsening or improvement in portal inflammation. Furthermore, all subjects showed improvement in Z-AAT burden, which was assessed by PAS+D (Periodic acid-Schiff with diastase) staining as shown in the following Table 7.

TABLE 7

Histological Assessment of Z-AAT Polymer (“Globules”) In The Liver From Baseline and Week 24 In Subjects From Cohort 1.

| | | |
|---|---|---|
| | BASELINE | WEEK 24 |
| Subject 1 | Zone 1 and 2 involvement | Zone 1 only |
| | More than ⅔ portal tracts | Less than ⅓ portal tracts |
| | ⅓ to ⅔ periportal hepatocytes | Less than ⅓ periportal hepatocytes |
| | Largest globule approximately equal to red blood cells (“RBC”) | Largest globule smaller than RBC |
| | Globules larger than RBC comprised< 10% of total granules | None larger than RBC |
| Subject 2 | Zone 1 only | No globules |
| | Less than ⅓ portal tracts | |
| | Less than ⅓ periportal hepatocytes | |
| | Largest globule approximately equal to RBC | |
| Subject 3 | Zones 1 and 2 | Unchanged |
| | More than ⅔ portal tracts | Less than ⅓ portal tracts |
| | More than ⅔ periportal hepatocytes | Less than ⅓ periportal hepatocytes |
| | Largest globule approximately equal to RBC | Largest globule smaller than RBC |
| | Globules larger than RBC comprised <10% of total granules | Unchanged |
| Subject 4 | All Zones, or only zone 2 and 3 | Zone 1 and 2 only |
| | More than ⅔ portal tracts | Unchanged |
| | More than ⅔ periportal hepatocytes | ⅓ to ⅔ periportal hepatocytes |
| | Largest globule approximately equal to RBC | Unchanged |
| | None larger than RBC | Globules larger than RBC comprise <10% of total granules |

Serum samples taken from the subjects in Cohort 1 at week 52 showed that similar declines in ALT, GGT, and serum Z-AAT levels from baseline to those shown in Table 6 at week 24 were still maintained.

### Cohort 2

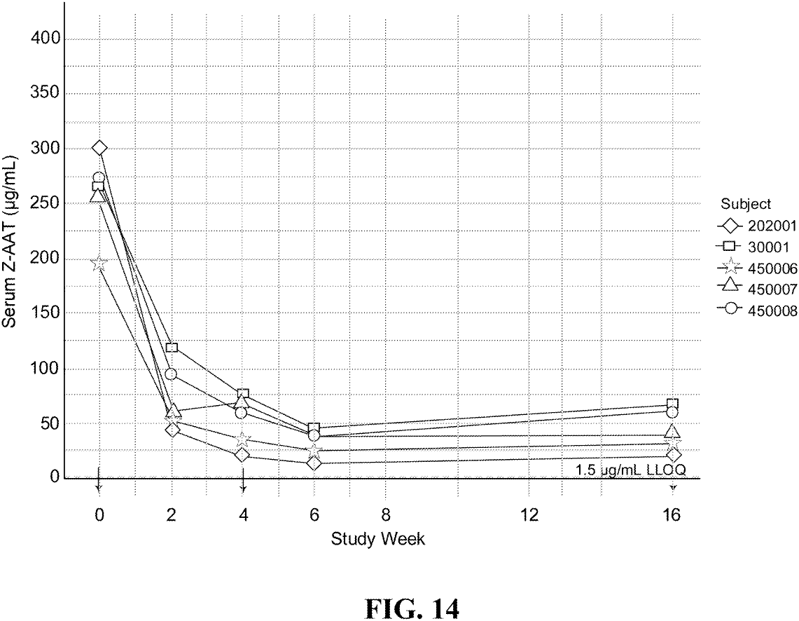

For Cohort 2, preliminary serum Z-AAT reduction results through week 16 for five patients are shown in FIG. 14, which showed reductions in Z-AAT protein levels similar to those in Cohort 1 at Week 24.

At week 48, the following Table 8 shows, among other things, total liver (i.e., intra-hepatic) Z-AAT protein levels, monomer (soluble) liver Z-AAT protein levels, polymer (insoluble) liver Z-AAT protein levels, ALT enzyme levels, and GGT enzyme levels, for the first 5 subjects in Cohort 2 that reached the 48 week timepoint. FIBROSCAN® assessments were taken for each of the subjects both pre-dose liver biopsy and at week 48, results of which are shown in Table 8 as well.

TABLE 8

Relative Change From Baseline to Week 48 In Subjects From Cohort 2.
Subject Number

| | | | | | |
|---|---|---|---|---|---|
| Pharmacodynamic Response | 1 | 2 | 3 | 4 | 5 |
| Δ % Serum Z-AAT | -96.7% | -79.4% | -86.1% | -86.7% | -90.3% |
| Total liver Z-AAT baseline^{∗}, | 64.1 | 60.2 | 23.0 | 31.9 | 83.7 |
| Δ nmol/total protein, | -57.5 | -48.2 | -20.6 | -24.6 | -81.2 |
| Δ % | -89.7% | -80.1% | -89.4% | -77.0% | -97.0% |
| Soluble liver Z-AAT baseline^{∗}, | 21.8 | 19.7 | 14.5 | 22.7 | 25.9 |
| Δ nmol/total protein, | -21.1 | -15.5 | -12.7 | -20.7 | -25.1 |
| Δ % | -96.7% | -78.5% | -87.8% | -91.1% | -97.0% |
| Δ Insoluble liver Z-AAT^{∗}, | -36.5 | -32.7 | -7.8 | -3.9 | -56.1 |
| Δ % | -86.2% | -80.8% | -92.1.% | -42.2% | -97.1% |
| Δ % ALT | -50.0%** | -42.6% | -34.5% | -60.8%** | -51.5% |
| Δ % GGT | -34.4% | -43.9% | -40.4% | -25.7% | -29.4% |
| Δ % FibroScan® | -17.7% | -17.0% | NM | -67.8% | -35.6% |
| Δ Pro-C3, ng/mL | -8.6 | -2.4 | -4.2 | -5.8 | -5.7 |
| Δ% | -36.6% | -17.6% | -25.5% | -28.3% | -30.8% |

^{∗}nmol/total protein
^{∗∗}Week 40 measurement
NM - not measured

As shown in Table 8 above, each of the reported subjects attained a greater than 75% reduction in total liver Z-AAT protein levels (Δ %) and monomer (soluble) liver Z-AAT protein levels. Moreover, all the subjects showed a reduction in polymer (insoluble) liver Z-AAT protein level, with 4 of the 5 reported subjects demonstrating reductions in Z-AAT polymer (insoluble) protein level in the range of 80-97%, and one subject demonstrating a reduction of 42.4%.

The reductions in liver Z-AAT protein levels also yielded improvements in clinically relevant biomarkers including that all subjects showed reductions in ALT ranging from 34-61%, and all subjects showed GGT reductions ranging from 26-44% compared to baseline at week 48.

All reported subjects showed an improvement in FIBROSCAN® score, with reductions of 17.7%, 17.0%, 67.8%, and 35.6%, respectively (only 4 of the 5 subjects were measured). Moreover, all 5 patients demonstrated reductions in the fibrogenesis biomarker Pro-C3 ranging from 18-37% at Week 48.

At week 48, 4 of 5 subjects showed at least 1 point improvement in fibrosis (METAVIR) with no worsening of fibrosis seen in the 5^{th} subject. Three of 5 subjects had improvement in portal inflammation, with the other 2 subjects showing no worsening. Furthermore, all subjects had improvement in Z-AAT burden, which was assessed by PAS+D (Periodic acid-Schiff with diastase) staining.

### Histological Assessment of Cohorts 1 and 2

Histological assessments of the liver biopsies at week 24 (for Cohort 1) and week 48 (for Cohort 2) were performed in a blinded fashion (to patient and time point) by two independent pathologists with a third pathologist involved when the assessments between the first two pathologists diverged. Fibrosis stage was scored by METAVIR and Z-AAT globule burden based on positive PAS+D staining scored 0-3 (3 being most severe or greatest burden) for each of the following: the extent of portal tracts containing globules, extent of periportal hepatocytes with globules, and zonal location. Key histological features that improved by greater or equal to 1-point are reported in the following Table 9:

TABLE 9

Liver Histology Improvement Over Baseline (Percentage of Subjects with ≥ 1 Point Improvement in Liver Histological Features).
≥ 1 Point Improvement

| Key Histological Feature | Cohort 1 (Week 24) N=4 | Cohort 2 (Week 48) N=5 | Total N=9 |
|---|---|---|---|
| Fibrosis (Metavir) | 50% (2/4) | 80% (⅘) | 67% (6/9) |
| Portal Inflammation | 67% (⅔^{∗∗∗}) | 60% (⅗) | 63% (⅝^{∗∗∗}) |
| Interface Hepatitis | 0% (0/3^{∗∗∗}) | 80% (⅘) | 50% (4/8^{∗∗∗}) |
| Global Portal Tract Involvement | 75% (¾) | 100% (5/5) | 89% (8/9) |
| PAS+D Zonal Location | 75% (¾) | 100% (5/5) | 89% (8/9) |
| Zone 1 “Globule” Periportal Involvement | 100% (4/4) | 100% (5/5) | 100% (9/9) |

^{∗∗∗} Excludes subject with no evidence at baseline

The available data from Cohort 1 and 2 demonstrate that ARO-AAT treatment led to a consistent and substantial reduction in intra-hepatic Z-AAT protein, both Z-AAT monomer and Z-AAT polymer; a consistent decrease in histological globule burden; improvements in fibrosis; and improvements in other relevant biomarkers of liver health. Additionally, histological improvement in steatosis was seen in subjects with baseline steatotic liver disease.

### Study Safety Summary

In the Phase II study, through the data extraction date of Mar. 15, 2021, there were 16 subjects that had received a total of 71 doses (59 doses of 200 mg and 12 doses of 100 mg) with no deaths and no dropouts due to adverse events. ARO-AAT was generally well tolerated after up to 1 year of treatment. There were no Treatment Emergent Adverse Events (TEAE)-related study drug discontinuation, dose interruptions, or premature study withdrawals. Three Serious Adverse Events (SAEs) of viral myocarditis, diverticulitis, and dyspnoea were reported in the 200 mg cohort. All were moderate in severity and all resolved. Viral myocarditis was associated with EBV infection, diverticulitis occurred in a subject with risk factors - a 63 yr old with PiZZ genotype and a history of appendectomy, and dyspnoea occurred in subject with medical history of non-obstructive pulmonary emphysema and delayed pulmonary care. The AAT RNAi Drug Substance has been well tolerated without any definitive safety signal, including no clinically meaningful reductions in ppFEV1 (percent predicted forced expiratory volume in one second) being observed from baseline to Week 48.

### Updated Compiled Data Summary of Cohorts 1, 2, and 1b Through Data Extraction Date of August 30, 2021

As described above, a total of 16 homozygous PiZZ subjects participated in the Phase 2 clinical trial. Cohorts 1 and 1b consisted of 4 subjects receiving a total of three doses of AAT RNAi Drug Substance at either 200 mg (Cohort 1) or 100 mg (Cohort 1b) administered as a subcutaneous injection, with doses administered on Day 1, four weeks after the initial dose, and twelve weeks after the second dose. Cohort 2 consisted of 8 subjects that received a total of five doses of AAT RNAi Drug Substance at 200 mg administered as a subcutaneous injection, with doses are administered on Day 1, four weeks after the initial dose, and twelve weeks after the second dose, twelve weeks after the third dose, and twelve weeks after the fourth dose (i.e., Day 1, and weeks 4, 16, 28, and 40). Mean age was 52 years (range 20-66 years), with 14 of the 16 subjects being men.

Paired biopsies collected at baseline and post-baseline (weeks 24 and 48 for Groups 1 and 2, and week 24 for Group 1b), and as of the cutoff data extraction date of Aug. 30, 2021 paired biopsies were available for 14 of the 16 subjects. Histology was assessed and adjudicated by 3 pathologists blinded to subject and time point. Key endpoints included METAVIR fibrosis, liver Z-AAT levels, and total globule burden (Sum of PAS+D staining for extent of portal tract and zone 1 periportal hepatocyte involvement and zonal location).

For the 14 subjects with paired biopsies, 11 had a METAVIR fibrosis stage of greater than or equal to F2 at baseline. ARO-AAT substantially reduced serum Z-AAT protein in all patients after the first dose, which was sustained throughout the observation. Mean percent reduction in total liver Z-AAT protein ranged from 80% to 89% at week 24 or week 48. All patients had reduced globule burden (mean score 7.3 (of a maximum of 9) at baseline that decreased to a mean score of 2.5 at week 24 or week 48). Improvement in fibrosis (≥ 1-stage) was achieved in six of 11 patients that were administered with 200 mg of AAT RNAi Drug Substance (i.e., subjects from Cohort 1 and Cohort 2), while none of three patients administered the 100 mg dose (Cohort 1b) showed improvement. While two patients in Cohort 2 showed a worsening of fibrosis from baseline to week 48 (both from F2 to F3), both subjects nevertheless had profound reductions in globule burden (scores of 9 and 4 at baseline, respectively, to both having scores of 0 at week 48), and normalized ALT and GGT levels after treatment.

All groups showed normalization of ALT and GGT. Mean percent reduction ranged from 42% to 56% for ALT and 33% to 54% between week 28 and week 72.

ARO-AAT was well tolerated, with no sustained clinically meaningful changes from baseline in ppFEV1 and no adverse events leading to study or study drug discontinuation. Four SAEs were reported: EBV-related myocarditis, diverticulitis, dyspnea, and vestibular neuronitis.

Certain summary data are provided in the following Table 10:

TABLE 10

Combined Cohort Summary Data Through Aug. 31, 2021 Data Cutoff

| | ARO-AAT 200 mg | | ARO-AAT 100 mg |
|---|---|---|---|
| | Group 1 (N=4) | Group 2 (N=8) | Group 1b (N=4) |
| Mean % Change (SD) Serum Z-AAT | | | |
| Week 24 (Cohort 1, 1b) / 28 (Cohort 2) | -89.5% (3.7) | -85.1% (7.8) | -83.9% (5.4) |
| Week 52 | -87.0% (7.3) | -82.4% (10.0) | NA |
| Week 72 | -92.0% (3.3) | NA | NA |
| Mean % Change (SD) Total Liver Z-AAT | | | |
| Week 24 | -79.8% (10.5) | NA | -83.1% (5.8) |
| Week 48 | NA | -88.8% (9.0) | NA |
| PAS+D Total Globule Burden, n/N (%) | | | |
| ≥ 1-point Improvement from BL to Week 24 or 48 | 4/4 (100%) | 7/7 (100%) ^{∗} | 4/4 (100%) |
| No Change from BL to Week 24 or 48 | 0/4 (0%) | 0/7 (0%) | 0/4 (0%) |
| METAVIR Fibrosis Stage, n/N (%) | | | |
| ≥ 1-point Improvement from Baseline to Week 24 or 48 | 2/4 (50%) | 4/7 (57.1%) | 0/3 (0%) ^{∗∗} |
| No Change from Baseline to Week 24 or 48 | 2/4 (50%) | ⅐ (14.3%) | 3/3 (100%) |
| ≥ 1-point Worsening from BL to Week 24 or 48 | 0/4 (0%) | 2/7(28.6%) | 0/3 (0%) |

NA = not applicable; liver biopsies were collected at Week 24 for Groups 1 and 1b, and at Week 48 for Group 2.
^{∗} At data cutoff, one subject in Group 2 had not yet reached Week 48 and was not included in the analysis
^{∗∗} One subject in Group 1b had baseline biopsy that was not evaluable for METAVIR fibrosis.

In sum, ARO-AAT reduced serum and liver Z-AAT and globule burden in all patients. These data demonstrate that removal of the causative factor, Z-AAT, in AATD liver disease ameliorates disease activity, and can lead to an improvement in fibrosis.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing liver Z-AAT protein levels in a human subject with a PiZZ genotype of alpha-1 antitrypsin, the method comprising:
a. administering to the subject an initial dose of a pharmaceutical composition that comprises the AAT RNAi Drug Substance described in Table 2 at a dose of between about 5 mg to about 300 mg of the AAT RNAi Drug Substance,
b. administering to the subject a second dose of the pharmaceutical composition about four weeks or about one month after the initial dose, and
c. administering to the subject a third dose of the pharmaceutical composition about twelve weeks or about three months after the second dose,
wherein the doses are administered by subcutaneous injection.

2. The method of claim 1, wherein the dose of the AAT RNAi Drug Substance is between about 25 mg and about 300 mg.

3. The method of claim 1, wherein the dose of the AAT RNAi Drug Substance is between about 25 mg and about 200 mg.

4. The method of claim 1, wherein the dose of the AAT RNAi Drug Substance is between about 100 mg and about 200 mg.

5. The method of claim 1, wherein the dose of the AAT RNAi Drug Substance is about 100 mg.

6. The method of claim 1, wherein the dose of the AAT RNAi Drug Substance is about 200 mg.

7. The method of claim 1, wherein the dose of the AAT RNAi Drug Substance is about 200 mg or less.

8. The method of any one of claims 1-7, wherein soluble liver Z-AAT protein level is reduced.

9. The method of any one of claims 1-8, wherein insoluble liver Z-AAT protein level is reduced.

10. The method of any one of claims 1-9, wherein both insoluble liver Z-AAT protein level and soluble liver Z-AAT protein level is reduced.

11. The method of any one of claims 1-10, further comprising administering additional doses after the third dose, wherein the additional doses are administered about every twelve weeks or about every three months thereafter.

12. The method of any one of claims 1-11, wherein the liver Z-AAT protein level is reduced within six months from the initial dose.

13. The method of any one of claims 1-12, wherein the liver Z-AAT protein level is reduced within about one year from the initial dose.

14. The method of any one of claims 1-13, wherein the Z-AAT protein level is reduced after the administration of only three doses of AAT RNAi Drug Substance.

15. The method of any one of claims 1-14, wherein the liver shows no worsening or an improvement in fibrosis.

16. The method of any one of claims 1-15, wherein liver enzymes of ALT, GGT, or both are reduced.

17. The method of any one of claims 1-16, wherein fibrogenesis marker Pro-C3 is reduced.

18. The method of any one of claims 1-17, wherein portal liver inflammation is reduced.

19. The method of any one of claims 1-18, wherein non-invasive measurement of liver stiffness by transient elastography (FibroScan®) is improved.

20. The method of any one of claims 1-19, wherein the subject is further administered an additional therapeutic for the treatment of AATD.

21. The method of any one of claims 1-20, wherein the subject is further administered a therapeutic for the treatment of lung damage, emphysema, or other lung diseases or disorders caused by the deficiency of endogenously secreted AAT protein.

22. The method of claim 21, wherein the additional therapeutic comprises human AAT protein, purified human alpha-1 proteinase inhibitor, or recombinant AAT protein.

23. The method of any one of claims 1-22, wherein the pharmaceutical composition is packaged in a kit, container, pack, dispenser, pre-filled syringe, or vials.

24. The method of any one of claims 1-23, wherein the pharmaceutical composition comprises, consists of, or consists essentially of the Formulated AAT RNAi Drug Substance described in Table 3.1 or Table 3.2.

25. The method of any one of claims 1-24, wherein the administration of one or more doses of the pharmaceutical composition is performed by the subject.

26. The method of any one of claims 1-25, wherein the administration of one or more doses of the pharmaceutical composition is performed by a medical professional.

27. The method of any one of claims 1-26, wherein the subject is an adult.

28. A method of treating AATD in a human subject with a PiZZ genotype of alpha-1 antitrypsin, the method comprising:
a. administering to the subject an initial dose of a pharmaceutical composition that comprises the AAT RNAi Drug Substance described in Table 2 at a dose of between about 5 mg to about 300 mg of the AAT RNAi Drug Substance,
b. administering to the subject a second dose of the pharmaceutical composition about four weeks or about one month after the initial dose, and
c. administering to the subject a third dose of the pharmaceutical composition about twelve weeks or about three months after the second dose,
wherein the doses are administered by subcutaneous injection.

29. The method of claim 28, wherein the condition or disease caused by AATD is a liver disease.

30. The method of claim 29, wherein the liver disease is chronic hepatitis, cirrhosis, increased risk of hepatocellular carcinoma, transaminitis, cholestasis, fibrosis, or fulminant hepatic failure.

31. The method of any one of claims 28-30, wherein the dose of the AAT RNAi Drug Substance is between about 100 mg and about 200 mg.

32. The method of any one of claims 28-30, wherein the dose of the AAT RNAi Drug Substance is about 200 mg or less.

33. The method of any one of claims 28-32, wherein monomer (soluble) liver Z-AAT protein level is reduced.

34. The method of any one of claims 28-33, wherein insoluble liver Z-AAT protein level is reduced.

35. The method of any one of claims 28-34, wherein both insoluble liver Z-AAT protein level and soluble liver Z-AAT protein level is reduced.

36. The method of any one of claims 28-35, further comprising administering additional doses after the third dose, wherein the additional doses are administered about every twelve weeks or about every three months thereafter.

37. The method of any one of claims 28-36, wherein the liver Z-AAT protein level is reduced within about six months of the initial dose.

38. The method of any one of claims 28-36, wherein the liver Z-AAT protein level is reduced within about one year of the initial dose.

39. The method of any one of claims 28-38, wherein the Z-AAT protein level is reduced after the administration of only three doses of AAT RNAi Drug Substance.

40. The method of any one of claims 1 to 39 wherein the administration of the pharmaceutical composition comprising the AAT RNAi Drug Substance described in Table 2 (ADS-001) to the human subject results in
(i) reduction in fibrosis;
(ii) reduction in level of periportal hepatocytes;
(iii) reduction in serum Z-AAT;
(iv) reduction in total liver Z-AAT;
(v) reduction in soluble liver Z-AAT;
(vi) reduction in insoluble liver Z-AAT;
(vii) reduction in ALT;
(viii) reduction in GGT;
(ix) reduction in Pro-C3;
(x) histological improvement in steatosis, or,
(xi) a combination thereof.

41. The method of claim 40, wherein the reduction in serum Z-AAT is at least about 70%.

42. The method of claim 40, wherein the reduction in serum Z-AAT is between about 70% and about 100%.

43. The method of claim 40, wherein the reduction in total liver Z-AAT is at least about 70%.

44. The method of claim 40, wherein the reduction in total liver Z-AAT is between about 70% and about 100%.

45. The method of claim 40, wherein the reduction in soluble liver Z-AAT is at least about 50%.

46. The method of claim 40, wherein the reduction in soluble liver Z-AAT is between about 50% and about 97%.

47. The method of claim 40, wherein the reduction in insoluble liver Z-AAT is at least about 40%.

48. The method of claim 40, wherein the reduction in insoluble liver Z-AAT is between about 40% and about 97%.

49. The method of claim 40, wherein the reduction in ALT is at least about 30%.

50. The method of claim 40, wherein the reduction in ALT is between about 30% and about 75%.

51. The method of claim 40, wherein the reduction in GGT is at least about 25%.

52. The method of claim 40, wherein the reduction in GGT is between about 25% and about 85%.

53. The method of claim 40, wherein the reduction in fibrosis is at least about 15% as measured by FIBROSCAN®.

54. The method of claim 40, wherein the reduction in fibrosis is between about 15% and about 90% as measured by FIBROSCAN®.

55. The method of claim 40, wherein the reduction in Pro-C3 is at least about 15%.

56. The method of claim 40, wherein the reduction in Pro-C3 is between about 15% and about 90%.

57. The method of claim 40, wherein the human subject has a histological improvement in steatosis.

58. The method of any one of claims 1 to 57 wherein the administration of the pharmaceutical composition comprising the AAT RNAi Drug Substance described in Table 2 (ADS-001) to the human subject results in improvements in fibrosis, Portal Inflammation, Interface Hepatitis, Global Portal tract Involvement, PAS+D Zonal Location, Zone 1 “Globule” Periportal Involvement, or any combination thereof.
